(19) United States
(12) Patent Application Publication
(10) Pub. No.: US 2015/0147356 A1
(43) Pub. Date: May 28, 2015

(54) ANTIPYRETICS TO ENHANCE TOLERABILITY OF VESICLE-BASED VACCINES

(71) Applicants: Alan Kimura, Hopkinton, MA (US); Peter Dull, Seattle, WA (US)

(72) Inventors: Alan Kimura, Hopkinton, MA (US); Peter Dull, Seattle, WA (US)

(21) Appl. No.: 14/117,280

(22) PCT Filed: May 11, 2012
(86) PCT No.: PCT/IB12/52354

§ 371 Date: Dec. 17, 2014

Related U.S. Application Data
(60) Provisional application No. 61/485,450, filed on May 12, 2011.

(51) Int. Cl. A61K 39/095; A61K 45/06; A61K 35/74

(52) U.S. Cl. CPC A61K 39/095; A61K 35/74; A61K 45/06
USPC 424/250.1; 424/282.1

(57) ABSTRACT

A method for immunising a human subject, wherein the subject receives (i) an immunogenic composition comprising bacterial vesicles and (ii) an antipyretic, and wherein the immunogenic composition and the antipyretic are administered to the subject within 24 hours of each other. Paracetamol significantly reduces fever rates without negatively affecting the immunogenicity either of a meningococcal vesicle vaccine or of concomitantly-administered antigens.

This application claims the benefit of U.S. provisional patent application 61/485,450 filed May 12th 2011, the complete contents of which are incorporated herein by reference for all purposes.

### TECHNICAL FIELD

This invention is in the field of vaccines based on membrane vesicles.

### BACKGROUND ART

Various vaccines against Neisseria meningitidis are currently being investigated. Some of these are based on outer membrane vesicles (OMVs), such as the Novartis MENZB™ product, the Finlay Institute VA-MENGOC-BC™ product, and the Norwegian Institute of Public Health MENBVAC™ product. After receiving these OMV-based vaccines, however, there have been some reports of fever in infants e.g. reference 1 mentions “frequently reported local reactions and fever in those under 5 years” even though the tested vaccines were “considered safe for use in all age groups”.

It is an object of the invention to provide ways of reducing the potential incidence of fever in subjects (particularly infants) who receive vesicle vaccines, but without having a negative impact on the vaccines' efficacy.

### DISCLOSURE OF THE INVENTION

According to the invention, subjects who receive a vesicle vaccine also receive an antipyretic. Although previous studies have reported that antipyretics can reduce vaccine-induced fever, they have also shown that this effect is accompanied by a loss of vaccine efficacy. For instance, reference 2 confirmed that “febrile reactions significantly decreased” when vaccinees received paracetamol (acetaminophen) but it also noted that “antibody responses to several vaccine antigens were reduced”, and reference 3 reports that these findings “present a compelling case against routine use of paracetamol during paediatric immunisations”. Reduced responses to Hib, diphtheria, tetanus and pertussis antigens had been observed in reference 2, and the same research group later confirmed in reference 4 that “prophylactic use of paracetamol reduced post-vaccination anti-pneumococcal antibody concentrations”. Furthermore, an earlier report [5] had failed to “find evidence that prophylaxis with acetaminophen or ibuprofen offers a clinically significant benefit in prevention of local reactions” to a fifth childhood immunisation. Similarly, reference 6 concludes that neither acetaminophen or ibuprofen “can be recommended prophylactically to prevent vaccine-associated adverse reactions”.

In contrast to this line of recent research, which points away from the administration of antipyretics when administering childhood vaccines, the results herein show that paracetamol significantly reduces fever rates without negatively affecting the immunogenicity either of a meningococcal vesicle vaccine or of concomitantly-administered antigens. Thus an antipyretic and an immunogenic composition comprising bacterial vesicles can both safely be administered to a subject. Preferably the antipyretic is administered (i) no more than 3 hours before the vesicles (ii) at the same time as the vesicles or (iii) no more than 2 hours after the vesicles.

Thus the invention provides a method for immunising a human subject, wherein the subject receives (i) an immunogenic composition comprising bacterial vesicles and (ii) an antipyretic, and wherein the immunogenic composition and the antipyretic are administered to the subject within 24 hours of each other.

The invention also provides a method for immunising a human subject, wherein the subject (i) receives an immunogenic composition comprising bacterial vesicles and (ii) has received an antipyretic no more than 24 hours before receiving the immunogenic composition.

The invention also provides a method for immunising a human subject, wherein the subject (i) receives an immunogenic composition comprising bacterial vesicles and (ii) has circulating antipyretic.

The invention also provides an immunogenic composition comprising bacterial vesicles and an antipyretic for combined use in a method of immunising a human subject, wherein the immunogenic composition and the antipyretic are administered to the subject within 24 hours of each other.

The invention also provides an immunogenic composition comprising bacterial vesicles and an antipyretic for combined use in a method of immunising a human subject as defined above.

The invention also provides the use of bacterial vesicles in the manufacture of an immunogenic composition for administering to a human subject within 24 hours of administering an antipyretic to the subject.

The invention also provides the use of bacterial vesicles in the manufacture of an immunogenic composition for administering to a human subject who has received an antipyretic no more than 24 hours earlier.

The invention also provides the use of bacterial vesicles in the manufacture of an immunogenic composition for administering to a human subject who has circulating antipyretic.

The invention also provides the use an antipyretic in the manufacture of a medicament for administering to a human subject within 24 hours of administering an immunogenic composition bacterial vesicles to the subject.

The invention also provides the use of (i) bacterial vesicles and (ii) an antipyretic, in the manufacture of a medicament for administering to a human subject within 24 hours of each other.

The invention also provides, in a method for immunising a human subject by administering an immunogenic composition comprising bacterial vesicles, an improvement consisting of administering an antipyretic to the subject within 24 hours of administering the immunogenic composition.

The invention also provides a combination of (i) an antipyretic and (ii) an immunogenic composition comprising bacterial vesicles, for simultaneous, separate or sequential administration, wherein components (i) and (ii) are administered within 24 hours of each other.

The invention also provides a combination of (i) an antipyretic and (ii) an immunogenic composition comprising bacterial vesicles, for separate or sequential administration, wherein components (i) and (ii) are administered within 24 hours of each other

The invention also provides a kit comprising (i) an antipyretic and (ii) an immunogenic composition comprising bacterial vesicles.

The invention also provides a package comprising (i) an immunogenic composition comprising bacterial vesicles for administering to a subject and (ii) an information leaflet containing written instructions that an antipyretic may be administered to a subject within 24 hours of their receiving the immunogenic composition.

The invention also provides a package comprising (i) an immunogenic composition comprising bacterial vesicles for administering to a subject and (ii) an information leaflet instructing a subject or physician to administer an antipyretic to the subject if the subject develops a fever after receiving the immunogenic composition.

The invention also provides a package comprising (i) an immunogenic composition comprising bacterial vesicles for administering to a subject and (ii) an information leaflet containing written instructions that an antipyretic should be administered to a subject within 24 hours of their receiving the immunogenic composition. The instructions can apply regardless of any fever development by the subject.

The invention also provides a package comprising (i) an immunogenic composition comprising bacterial vesicles for administering to a subject and (ii) an information leaflet instructing the physician that an antipyretic should be administered to a subject within 24 hours of their receiving the immunogenic composition. These instructions can apply regardless of any fever development by the subject.

### The Human Subject

The invention is useful for immunising human subjects. It can be used with children and adults, and so the subject may be less than 1 year old, 1-5 years old, 2-11 years old, 5-15 years old, 12-21 years old, 15-55 years old, or at least 55 years old. Reducing fever in infants and toddlers is of particular interest, and so the subject is preferably less than 1 year old (e.g. between 0-6 months old) or is between 1-5 years old.

The subject can be in any ethnic or racial group.

The subject may already have received at least one previous vaccine. Thus the subject's immune system may have been previously exposed to vaccine antigens e.g. to diphtheria toxoid (Dt), tetanus toxoid (Tt). Thus the subject may previously have raised an anti-Dt antibody response (typically to give an anti-Dt titer >0.01 IU/ml) and will possess memory B and/or T lymphocytes specific for Dt. Similarly, the subject may previously have raised an anti-Tt antibody response (typically to give an anti-Tt titer >0.01 IU/ml) and will possess memory B and/or T lymphocytes specific for Tt. Thus the subject may be distinct from subjects in general, as they are members of a subset of the general population whose immune systems have already mounted an immune response to e.g. Dt and/or Tt. As well as having been previously exposed to Dt or Tt, the subject may previously have received other antigens e.g. pertussis antigen(s), Haemophilus influenzae type B capsular saccharide, hepatitis B virus surface antigen (HBsAg), inactivated poliovirus vaccine, Streptococcus pneumoniae capsular saccharides, influenza virus vaccine, BCG, measles virus, mumps virus, rubella virus, varicella virus, N.meningitidis capsular saccharide(s), etc.

In some embodiments the subject has received an antipyretic no more than 24 hours before receiving the immunogenic composition. A subject who has taken an antipyretic will still have antipyretic circulating in their blood at a level which can exert a therapeutic effect when the immunogenic composition is administered. Assays for blood levels of antipyretics are well known in the art e.g. for checking for overdoses. Therapeutic blood levels of common antipyretics are as follows [7]:

| Antipyretic | Therapeutic blood level |
|---|---|
| Acetaminophen | 10-30 μg/ml (66-199 μM) |
| Ibuprofen | 10-50 μg/ml (49-243 μM) |
| Salicylates | 150-300 μg/ml (1086-2172 μM) |

### The Bacterial Vesicles

Although most clinical experience with vesicle vaccines is based on meningococcus, vesicle-based vaccines are also known for further Gram-negative bacteria.

Thus the vesicles may be from a species in any of genera Escherichia, Shigella, Neisseria, Moraxella, Bordetella, Borrelia, Brucella, Chlamydia Haemophilus, Legionella, Pseudomonas, Yersinia, Helicobacter, Salmonella, Vibrio, etc. For example, the vesicles may be from Bordetella pertussis, Borrelia burgdorferi, Brucella melitensis, Brucella ovis, Chlamydia psittaci, Chlamydia trachomatis, Moraxella catarrhalis, Escherichia coli (including extraintestinal pathogenic strains), Haemophilus influenzae (including non-typeable stains), Legionella pneumophila, Neisseria gonorrhoeae, Neisseria meningitidis, Neisseria lactamica, Pseudomonas aeruginosa, Yersinia enterocolitica, Helicobacter pylori, Salmonella enterica (including serovar typhi and typhimurium), Vibrio cholerae, Shigella dysenteriae, Shigella flexneri, Shigella boydii or Shigella sonnei, etc.

The invention is particularly suitable for use with Neisseria meningitidis vesicles e.g. prepared from a serogroup B N. meningitidis. Reference 28 discloses other bacteria which can be used.

The vesicles can be prepared from a wild-type bacterium or from a modified bacterium e.g. a strain which has been modified to inactivate genes which lead to a toxic phenotype. For example, it is known to modify bacteria so that they do not express a native lipopolysaccharide (LPS), particularly for E. coli, meningococcus, Shigella, and the like. Various modifications of native LPS can be made e.g. these may disrupt the native lipid A structure, the oligosaccharide core, or the outer O antigen. Absence of O antigen in the LPS is useful, as is absence of hexa-acylated lipid A. Inactivation of enterotoxins is also known e.g. to prevent expression of Shiga toxin.

Vesicles useful with the invention are any proteoliposomic vesicle obtained by disruption of or blebbling from a Gram-negative bacterial outer membrane to form vesicles therefrom which retain antigens from the outer membrane. Thus the term includes, for instance, OMVs (sometimes referred to as ‘blebs’), microvesicles (MVs [8]) and ‘native OMVs’ (′NOMVs' [9]).

MVs and NOMVs are naturally-occurring membrane vesicles that form spontaneously during bacterial growth and are released into culture medium. MVs can be obtained by culturing bacteria in broth culture medium, separating whole cells from the smaller MVs in the broth culture medium (e.g. by filtration or by low-speed centrifugation to pellet only the cells and not the smaller vesicles), and then collecting the MVs from the cell-depleted medium (e.g. by filtration, by differential precipitation or aggregation of MVs, by high-speed centrifugation to pellet the MVs). Strains for use in production of MVs can generally be selected on the basis of the amount of MVs produced in culture e.g. refs. 10 & 11 describe Neisseria with high MV production.

OMVs are prepared artificially from bacteria, and may be prepared using detergent treatment (e.g. with deoxycholate), or by non-detergent means (e.g. see reference 12). Techniques for forming OMVs include treating bacteria with a bile acid salt detergent (e.g. salts of lithocholic acid, chenodeoxycholic acid, ursodeoxycholic acid, deoxycholic acid, cholic acid, ursocholic acid, etc., with sodium deoxycholate[13 & 14] being preferred for treating Neisseria) at a pH sufficiently high not to precipitate the detergent [15]. Other techniques may be performed substantially in the absence of detergent [12] using techniques such as sonication, homogenisation, microfluidisation, cavitation, osmotic shock, grinding, French press, blending, etc. Methods using no or low detergent can retain useful antigens such as NspA in meningococcus [12]. Thus a method may use an OMV extraction buffer with about 0.5% deoxycholate or lower e.g. about 0.2%, about 0.1%, <0.05% or zero.

A useful process for OMV preparation is described in reference 16 and involves ultrafiltration on crude OMVs, rather than instead of high speed centrifugation. The process may involve a step of ultracentrifugation after the ultrafiltration takes place.

Another useful process for outer membrane vesicle production is to inactivate the mltA gene in a meningococcus, as disclosed in reference 17. These mutant bacteria spontaneously release vesicles into their culture medium.

### Meningococcal Vesicles

The invention can be used with various types of vesicle which are known for Neisseria meningitidis. Reference 18 discloses the construction of vesicles from strains modified to express six different PorA subtypes. References 19-21 report pre-clinical studies of an OMV vaccine in which fHbp (also known as GN1870) is over-expressed (and this over-expression can be combined with knockout of LpxL1 [22]). Reference 23 recently reported a clinical study of five formulations of an OMV vaccine in which PorA & FrpB are knocked-out and Hsf & TbpA are over-expressed. Reference 24 reports a native outer membrane vesicle vaccine prepared from bacteria having inactivated synX, lpxL1, and lgtA genes.

OMVs can be prepared from meningococci which over-express desired antigen(s) due to genetic modification. In addition to genetic modification(s) which cause over-expression of antigen(s) of interest, the bacteria may include one or more further modifications. For instance, the bacterium may have a knockout of one or more of lpxL1, lgtB, porA, frpB, synX, lgtA, mltA and/or lst.

The bacterium may have low endotoxin levels, achieved by knockout of enzymes involved in LPS biosynthesis [25,26].

The bacterium may be from any meningococcal serogroup e.g. A, B, C, W135, Y (preferably B).

The bacterium may be of any serotype (e.g. 1, 2a, 2b, 4, 14, 15, 16, etc.), any serosubtype, and any immunotype (e.g. L1; L2; L3; L3,3,7; L10; etc.). Vesicles can usefully be prepared from strains having one of the following subtypes: P1.2; P1.2,5; P1.4; P1.5; P1.5,2; P1.5,c; P1.5c, 10; P1.7,16; P1.7,16b; P1.7h, 4; P1.9; P1.15; P1.9,15; P1.12,13; P1.13; P1.14; P1.21,16; P1.22,14.

The bacterium may be from any suitable lineage, including hyperinvasive and hypervirulent lineages e.g. any of the following seven hypervirulent lineages: subgroup I; subgroup III; subgroup IV-1; ET-5 complex; ET-37 complex; A4 cluster; lineage 3. These lineages have been defined by multilocus enzyme electrophoresis (MLEE), but multilocus sequence typing (MLST) has also been used to classify meningococci [ref. 27] e.g. the ET-37 complex is the ST-11 complex by MLST, the ET-5 complex is ST-32 (ET-5), lineage 3 is ST-41/44, etc.

In some embodiments a bacterium may include one or more of the knockout and/or hyper-expression mutations disclosed in references 42 and 28-30. Suitable genes for modification include: (a) Cps, CtrA, CtrB, CtrC, CtrD, FrpB, GalE, HtrB/MsbB, LbpA, LbpB, LpxK, Opa, Opc, PilC, PorB, SiaA, SiaB, SiaC, SiaD, TbpA, and/or TbpB [28]; (b) CtrA, CtrB, CtrC, CtrD, FrpB, GalE, HtrB/MsbB, LbpA, LbpB, LpxK, Opa, Opc, PhoP, PilC, PmrE, PmrF, SiaA, SiaB, SiaC, SiaD, TbpA, and/or TbpB; (c) ExbB, ExbD, rmpM, CtrA, CtrB, CtrD, GalE, LbpA, LpbB, Opa, Opc, PilC, PorB, SiaA, SiaB, SiaC, SiaD, TbpA, and/or TbpB; and (d) CtrA, CtrB, CtrD, FrpB, OpA, OpC, PilC, PorB, SiaD, SynA, SynB, and/or SynC.

A bacterium may have one or more, or all, of the following characteristics: (i) down-regulated or knocked-out LgtB and/or GalE to truncate the meningococcal LOS; (ii) up-regulated TbpA; (iii) up-regulated NhhA; (iv) up-regulated Omp85; (v) up-regulated LbpA; (vi) up-regulated NspA; (vii) knocked-out PorA; (viii) down-regulated or knocked-out FrpB; (ix) down-regulated or knocked-out Opa; (x) down-regulated or knocked-out Opc; (xi) deleted cps gene complex; (xi) up-regulated NHBA; (xii) up-regulated NadA; (xiii) up-regulated NHBA and NadA; (xiv) up-regulated fHbp; (xv) down-regulated LpxL1. A truncated LOS can be one that does not include a sialyl-lacto-N-neotetraose epitope e.g. it might be a galactose-deficient LOS. The LOS may have no α chain.

If lipo-oligosaccharide (LOS) is present in a vesicle it is possible to treat the vesicle so as to link its LOS and protein components (“intra-bleb” conjugation[30]).

The vesicles may lack LOS altogether, or they may lack hexa-acylated LOS e.g. LOS in the vesicles may have a reduced number of secondary acyl chains per LOS molecule [31]. For example, the vesicles may from a strain which has a lpxL1 deletion or mutation which results in production of a penta-acylated LOS [20,24]. LOS in a strain may lack a lacto-N-neotetraose epitope e.g. it may be a lst and/or lgtB knockout strain [23]. LOS may lack at least one wild-type primary O-linked fatty acid [32]. LOS having. The LOS may have no α chain. The LOS may comprise GlcNAc-Hep₂phosphoethanolamine-KDO₂-Lipid A [33].

As a result of up-regulation mentioned above, vesicles prepared from modified meningococci contain higher levels of the up-regulated antigen(s). The increase in expression in the vesicles (measured relative to a corresponding wild-type strain) is usefully at least 10%, measured in mass of the relevant antigen per unit mass of vesicle, and is more usefully at least 20%, 30%, 40%, 50%, 75%, 100% or more.

Suitable recombinant modifications which can be used to cause up-regulation of an antigen include, but are not limited to: (i) promoter replacement; (ii) gene addition; (iii) gene replacement; or (iv) repressor knockout. In promoter replacement, the promoter which controls expression of the antigen's gene in a bacterium is replaced with a promoter which provides higher levels of expression. For instance, the gene might be placed under the control of a promoter from a housekeeping metabolic gene. In other embodiments, the antigen's gene is placed under the control of a constitutive or inducible promoter. Similarly, the gene can be modified to ensure that its expression is not subject to phase variation. Methods for reducing or eliminating phase variability of gene expression in meningococcus are disclosed in reference 34. These methods include promoter replacement, or the removal or replacement of a DNA motif which is responsible for a gene's phase variability. In gene addition, a bacterium which already expresses the antigen receives a second copy of the relevant gene. This second copy can be integrated into the bacterial chromosome or can be on an episomal element such as a plasmid. The second copy can have a stronger promoter than the existing copy. The gene can be placed under the control of a constitutive or inducible promoter. The effect of the gene addition is to increase the amount of expressed antigen. In gene replacement, gene addition occurs but is accompanied by deletion of the existing copy of the gene. For instance, this approach was used in reference 21, where a bacterium's endogenous chromosomal fHbp gene was deleted and replaced by a plasmid-encoded copy (see also reference 35). Expression from the replacement copy is higher than from the previous copy, thus leading to up-regulation. In repressor knockout, a protein which represses expression of an antigen of interest is knocked out. Thus the repression does not occur and the antigen of interest can be expressed at a higher level. Promoters for up-regulated genes can advantageously include a CREN [36].

A modified strain will generally be isogenic with its parent strain, except for a genetic modification. As a result of the modification, expression of the antigen of interest in the modified strain is higher (under the same conditions) than in the parent strain. A typical modification will be to place a gene under the control of a promoter with which it is not found in nature and/or to knockout a gene which encodes a repressor.

In embodiments where NHBA is up-regulated, various approaches can be used. For convenience, the approach already reported in reference 37 can be used i.e. introduction of a NHBA gene under the control of an IPTG-inducible promoter. By this approach the level of expression of NHBA can be proportional to the concentration of IPTG added to a culture. The promoter may include a CREN.

In embodiments where NadA is up-regulated, various approaches can be used. One useful approach involves deletion of the gene encoding NadR (NMB1843), which is a transcriptional repressor protein [38] which down-regulates or represses the NadA-encoding gene in all strains tested. Knockout of NadR results in high-level constitutive expression of NadA. An alternative approach to achieve NadA up-regulation is to add 4-hydroxyphenylacetic to the culture medium. A further approach is to introduce a NadA gene under the control of an IPTG-inducible promoter.

Up-regulation of NhhA is already reported in references 23 and 39. Up-regulation of TbpA is already reported in references 23, 39 and 40. Up-regulation of HmbR is already reported in reference 41. Up-regulation of TbpB is already reported in reference 40. Up-regulation of NspA is already reported in reference 42, in combination with porA and cps knockout. Up-regulation of Cu,Zn-superoxide dismutase is already reported in reference 40. Up-regulation of fHbp is already reported in references 19-21 & 35, and by a different approach (expressing a constitutively-active mutant FNR) in references 43 & 44.

In some embodiments each of NHBA, NadA and fHbp are up-regulated. These three antigens are components of the “universal vaccine” disclosed in reference 45 or “4CMenB” [46,47]. In one embodiment, expression of NHBA is controlled by a strong promoter, NadR is knocked out, and the strain expresses a constitutively active mutant FNR. In another embodiment, expression of NHBA is controlled by a strong promoter, expression of fHbp is controlled by a strong promoter, and NadR is knocked out. The bacterium can also be a bacterium which does not express an active MltA (GNA33), such that it spontaneously releases vesicles which contain NHBA, NadA and fHbp. Ideally, the bacterium does not express a native LPS e.g. it has a mutant or knockout of LpxL1.

The vesicles may include one, more than one, or (preferably) zero PorA serosubtypes. Modification of meningococcus to provide multi-PorA OMVs is known e.g. from references 18 and 48. Conversely, modification to remove PorA is also known e.g. from reference 23.

The vesicles may be free from one of both of PorA and FrpB. Preferred vesicles are PorA-free.

The invention may be used with mixtures of vesicles from different strains. For instance, reference 49 discloses vaccine comprising multivalent meningococcal vesicle compositions, comprising a first vesicle derived from a meningococcal strain with a serosubtype prevalent in a country of use, and a second vesicle derived from a strain that need not have a serosubtype prevent in a country of use. Reference 50 also discloses useful combinations of different vesicles. A combination of vesicles from strains in each of the L2 and L3 immunotypes may be used in some embodiments.

One way of checking efficacy of therapeutic treatment involves monitoring meningococcal infection after administration of the composition of the invention. One way of checking efficacy of prophylactic treatment involves monitoring immune responses against meningococcal antigen(s) after administration of the composition. Immunogenicity of compositions of the invention can be determined by administering them to test subjects (e.g. children 12-16 months age, or animal models [51]) and then determining standard parameters including serum bactericidal antibodies (SBA) and ELISA titres (GMT). These immune responses will generally be determined around 4 weeks after administration of the composition, and compared to values determined before administration of the composition. A SBA increase of at least 4-fold or 8-fold is preferred. Where more than one dose of the composition is administered, more than one post-administration determination may be made.

In general, compositions of the invention are able to induce anti-meningococcal serum bactericidal antibody responses after being administered to a subject. These responses are conveniently measured in mice and are a standard indicator of vaccine efficacy. Serum bactericidal activity (SBA) measures bacterial killing mediated by complement, and can be assayed using human or baby rabbit complement. WHO standards require a vaccine to induce at least a 4-fold rise in SBA in more than 90% of recipients.

Preferred compositions can confer an anti-meningococcal antibody titre in a human subject that is superior to the criterion for seroprotection for an acceptable percentage of subjects. Antigens with an associated antibody titre above which a host is considered to be seroconverted against the antigen are well known, and such titres are published by organisations such as WHO. Preferably more than 80% of a statistically significant sample of subjects is seroconverted, more preferably more than 90%, still more preferably more than 93% and most preferably 96-100%.

### The Immunogenic Composition

The immunogenic composition can include further components in addition to the bacterial vesicles. These further components can include further immunogens and/or non-immunogens.

Thus the immunogenic composition will typically include a pharmaceutically acceptable carrier, and a thorough discussion of such carriers is available in reference 52.

The pH of the immunogenic composition is usually between 6 and 8, and more preferably between 6.5 and 7.5 (e.g. about 7). The pH of the RIVM OMV-based vaccine is 7.4 [53], and a pH<7.5 is preferred for compositions of the invention. The RIVM OMV-based vaccine maintains pH by using a 10 mM Tris/HCl buffer, and stable pH in compositions of the invention may be maintained by the use of a buffer e.g. a Tris buffer, a citrate buffer, phosphate buffer, or a histidine buffer. Thus immunogenic compositions of the invention will generally include a buffer.

The immunogenic composition may be sterile and/or pyrogen-free. The immunogenic composition may be isotonic with respect to humans.

Immunogenic compositions of the invention for administration to subjects are preferably vaccine compositions. Vaccines according to the invention may either be prophylactic (i.e. to prevent infection) or therapeutic (i.e. to treat infection), but will typically be prophylactic. Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials. The antigen content of compositions of the invention will generally be expressed in terms of the amount of protein per dose. The concentration of vesicles in compositions of the invention will generally be between 10 and 500 μg/ml, preferably between 25 and 200 μg/ml, and more preferably about 50 μg/ml or about 100 μg/ml (expressed in terms of total protein in the vesicles).

Immunogenic compositions may include an immunological adjuvant. Thus, for example, they may include an aluminium salt adjuvant or an oil-in-water emulsion (e.g. a squalene-in-water emulsion). Suitable aluminium salts include hydroxides (e.g. oxyhydroxides), phosphates (e.g. hydroxyphosphates, orthophosphates), (e.g. see chapters 8 & 9 of ref. 54), or mixtures thereof. The salts can take any suitable form (e.g. gel, crystalline, amorphous, etc.), with adsorption of antigen to the salt being preferred. The concentration of Al⁺⁺⁺ in a composition for administration to a subject is preferably less than 5 mg/ml e.g. ≦4 mg/ml, ≦3 mg/ml, ≦2 mg/ml, ≦1 mg/ml, etc. A preferred range is between 0.3 and 1 mg/ml. A maximum of 0.85 mg/dose is preferred. Aluminium hydroxide adjuvants are particularly suitable for use with meningococcal vaccines.

Bacteria such as meningococci affect various areas of the body and so the compositions of the invention may be prepared in various liquid forms. For example, the compositions may be prepared as injectables, either as solutions or suspensions. The composition may be prepared for pulmonary administration e.g. by an inhaler, using a fine spray. The composition may be prepared for nasal, aural or ocular administration e.g. as spray or drops, and intranasal vesicle vaccines are known in the art. Injectables for intramuscular administration are typical. Injection may be via a needle (e.g. a hypodermic needle), but needle-free injection may alternatively be used.

Compositions may include an antimicrobial, particularly when packaged in multiple dose format. Antimicrobials such as thiomersal and 2-phenoxyethanol are commonly found in vaccines, but it is preferred to use either a mercury-free preservative or no preservative at all.

Compositions may comprise detergent e.g. a Tween (polysorbate), such as Tween 80. Detergents are generally present at low levels e.g. <0.01%.

Compositions may include residual detergent (e.g. deoxycholate) from OMV preparation. The amount of residual detergent is preferably less than 0.4 μg (more preferably less than 0.2 μg) for every μg of vesicle protein.

If a composition includes LOS, the amount of LOS is preferably less than 0.12 μg (more preferably less than 0.05μg) for every μg of vesicle protein.

Compositions may include sodium salts (e.g. sodium chloride) e.g. for controlling tonicity. A concentration of 10±2 mg/ml NaCl is typical e.g. about 9 mg/ml.

Effective dosage volumes can be routinely established, but a typical human dose of the composition has a volume of about 0.5 ml e.g. for intramuscular injection (e.g. into the thigh or upper arm). The RIVM OMV-based vaccine was administered in a 0.5 ml volume [55] by intramuscular injection to the thigh or upper arm. McNZB™ is administered in a 0.5 ml by intramuscular injection to the anterolateral thigh or the deltoid region of the arm. Similar doses may be used for other delivery routes e.g. an intranasal OMV-based vaccine for atomisation may have a volume of about 100 μl or about 130 μl per spray, with four sprays administered to give a total dose of about 0.5 ml.

In addition to containing vesicles as an immunogenic component, the composition can include one or more further meningococcal protein immunogens. For instance, the composition can include a NHBA antigen, a fHbp antigen, and a NadA antigen. For instance, the BEXSERO™ product from Novartis can be used. This includes NadA, fHbp, NHBA and OMVs from a B:4:P1.7-2,4 epidemic strain [56]. Thus the composition may include OMVs, and three separate proteins comprising of amino acid sequences SEQ ID NOs 4, 5 and 6.

In addition to containing vesicles (and optional further proteins) as an immunogenic component, the composition can include one or more further meningococcal saccharide immunogens. For instance, the composition can include one or more capsular saccharides from meningococci e.g. from serogroups A, C, W135 and/or Y. These saccharides will usually be conjugated to a protein carrier e.g. to tetanus toxoid, diphtheria toxoid, or CRM197. A composition of the invention may include one or more conjugates of capsular saccharides from 1, 2, 3, or 4 of meningococcal serogroups A, C, W135 and Y e.g. A+C, A+W135, A+Y, C+W135, C+Y, W135+Y, A+C+W135, A+C+Y, A+W135+Y, A+C+W135+Y, etc. Components including saccharides from all four of serogroups A, C, W135 and Y are ideal. For instance, the immunogenic composition could be prepared by mixing vesicles with either the MENVEO™ or MENACTRA™ 4-valent A-C-W135-Y meningococcal conjugate vaccine. This approach is useful for preparing a 5-valent meningococcal which can protect against each of serogroups A, B, C, W135 and Y.

As well as containing the vesicles (and optional meningococcal saccharide conjugates), the immunogenic composition can include antigens from further pathogens e.g. one or more of:
an antigen from Streptococcus pneumoniae, such as a saccharide (typically conjugated)
an antigen from hepatitis B virus, such as the surface antigen HBsAg.
an antigen from Bordetella pertussis, such as pertussis holotoxin (PT) and filamentous haemagglutinin (FHA) from B.pertussis, optionally also in combination with pertactin and/or agglutinogens 2 and 3.
a diphtheria antigen, such as a diphtheria toxoid.
a tetanus antigen, such as a tetanus toxoid.
a saccharide antigen from Haemophilus influenzae B (Hib), typically conjugated.
inactivated poliovirus antigens, typically trivalent from polioviruses 1, 2 and 3.

### The Antipyretic

Antipyretics are pharmacological agents which reduce fever. They do not normally lower body temperature if the subject does not have a fever because, rather than causing a drop in body temperature, they instead cause the hypothalamus to override an interleukin-induced increase in normal body temperature.

The antipyretic may be a non-steroidal anti-inflammatory drug (NSAID) such as ibuprofen, naproxen sodium, ketoprofen, or nabumetone. The antipyretic may be a salicylate, such as an aspirin (acetylsalicylic acid), choline salicylate, magnesium salicylate, or sodium salicylate. The antipyretic may be paracetamol (acetaminophen). The antipyretic may be metamizole sodium or dipyrone. The antipyretic may be phenazone. The antipyretic may be quinine.

Two preferred antipyretics for use with the invention are acetaminophen or ibuprofen. The most preferred is acetaminophen as this has an established safety profile in infants.

In some embodiments a combination of antipyretics is used. For instance, it is known to administer a combination of acetaminophen and aspirin, or a combination of acetaminophen and ibuprofen. Where more than one antipyretic is used, these may be given at the same time (separately or in combination) or may be given at different times e.g. in alternating sequence.

Suitable dosing of antipyretics is known in the art e.g. acetaminophen can be administered at a dose of 10-15 mg per kg body weight (or 5 mg/kg in jaundiced children), ibuprofen can be administered at 7.5-10 mg/kg, etc.

### Administration of the Two Components

The invention involves administering to a human subject (i) an immunogenic composition comprising bacterial vesicles and (ii) an antipyretic. This may involve giving the vesicles and the antipyretic at the same time. Where the two components are not administered at the same time, though, they are administered within 24 hours of each other, in either order. Thus the invention may involve giving an antipyretic to a subject who has received an immunogenic composition, or may involve giving an immunogenic composition to a subject who has received an antipyretic.

The vesicles and antipyretic are administered within 24 hours of each other. Ideally they are administered within 12 hours of each other e.g. within 6 hours of each other, within 3 hours of each other, within 2 hours of each other, within 1 hour of each other, within 30 minutes of each other, within 20 minutes of each other, within 10 minutes of each other, or within 5 minutes of each other.

Where the antipyretic is administered before the immunogenic composition, the time difference between the administrations is ideally less than 4 hours (or even less than 2 hours), in order that the antipyretic is still circulating at effective levels. For instance, the half-life of acetaminophen is about 3 hours, and the duration of action of ibuprofen is about 4 hours, and so administration of the immunogenic composition within 4 hours of the antipyretic ensures that the subject is still benefitting from the therapeutic effect of the previously-administered antipyretic.

In a typical embodiment the antipyretic will be administered to the subject prophylactically before the vesicles e.g. no more than 60 minutes before, no more than 40 minutes before, no more than 30 minutes before, no more than 20 minutes before, no more than 10 minutes before, or no more than 5 minutes before. The antipyretic can be administered prophylactically to subjects in general, without necessarily determining whether any individual subject would receive specific benefit from the antipyretic, and without being administered in response to an observed fever.

In preferred embodiments the antipyretic is administered (i) no more than 3 hours before the vesicles, and ideally no more than 1 hour before, (ii) at the same time as the vesicles, or (iii) no more than 2 hours after the vesicles, and ideally no more than 1 hour after. This close timing of administration ensures that the antipyretic effect is given to the patient on a timescale suitable for any potential febrile reaction to the immunogenic composition.

The invention will typically involve only a single administration of vesicles within a 24 hours period, but it may involve more than one administration of antipyretic e.g. the invention may involve 1, 2, 3, 4 or more administrations of antipyretic. Where more than one antipyretic administration is given then the above timing (i.e. within 24 hours of each other, down to within 5 minutes of each other) refers to the shortest period between administration of a vesicle and administration of an antipyretic component. Overall, though, it is nevertheless feasible to achieve administration of vesicles and all antipyretic doses within 24 hours.

Where the invention does involve more than one administration of antipyretic, these (i) can all be before administration of the vesicles, (ii) can all be after administration of the vesicles, (iii) can span administration of the vesicles, with at least one before and at least one after, or (iv) can involve at least one administration before and/or after, together with one administration at the same time as the vesicles.

Where the invention does involve more than one administration of antipyretic, each separate administration can use the same antipyretic (or combination of antipyretics), but in some embodiments different antipyretics can be used e.g. an alternating sequence of acetaminophen and ibuprofen.

In a typical embodiment, the invention involves: (i) administration of an antipyretic; then (ii) within 20 minutes of step (i), administration of the immunogenic composition; then (iii) one or two further doses, and possibly a third, after step (ii). A maximum of 4 doses of antipyretic in a 24 hour period is typical. The first (or only) further dose given in step (iii) will typically be given 4-6 hours after step (ii), and any further dose(s) at 4-6 hour intervals.

Administration of the antipyretic and the immunogenic composition can be performed by a regimen comprising serial administration(s). A combination of the antipyretic and the immunogenic composition can be administered 2 or more times in series, e.g. 3 times in series. Each administration of the combination in a series can be administered within between 2 weeks and 6 months, e.g. 1 month, of the preceding administration of the combination in the series. For example a combination of the antipyretic and the immunogenic composition can be administered to a subject at 2 months of age, and then again at 3 months of age. The combination can be administered again at 4 months of age. The combination of the antipyretic and the immunogenic composition does not need to be administered identically each time. In each administration the antipyretic and the immunogenic composition of the combination are administered to the subject within 24 hours of each other.

Administration of the antipyretic and the immunogenic composition can be performed by the same person or by different people. The immunogenic composition will generally be administered by a healthcare professional (e.g. physician, nurse) whereas the antipyretic can be self-administered.

Generally, the immunogenic composition will be administered by injection (e.g. intramuscular injection) whereas the antipyretic will be administered orally (e.g. by tablet or capsule, or by liquid oral suspension).

### Administration of Further Components

The invention involves administering to a human subject (i) an immunogenic composition comprising bacterial vesicles and (ii) an antipyretic. As mentioned above, the immunogenic composition can include components in addition to the bacterial vesicles. Furthermore, the invention can involve administering more than just components (i) and (ii). For example, the subject might receive (i) a first immunogenic composition comprising bacterial vesicles, (ii) an antipyretic, and (iii) a second immunogenic composition which does not comprise bacterial vesicles.

Suitable second immunogenic compositions are common childhood vaccines e.g. comprising diphtheria toxoid, tetanus toxoid, cellular or acellular pertussis antigens, conjugated H. influenzae type B capsular saccharide, hepatitis B virus surface antigen, inactivated poliovirus antigens, conjugated N.meningitidis capsular saccharides from one or more of serogroups A, C, W135 &/or Y, an influenza virus vaccine, conjugated S.pneumoniae capsular saccharides a MMR vaccine, a rotavirus vaccine, a varicella vaccine, a hepatitis A virus vaccine, etc.

In one embodiment a subject receives (i) a first immunogenic composition comprising bacterial vesicles, (ii) an antipyretic, and (iii) a second immunogenic composition which is a combination vaccine comprising diphtheria toxoid, tetanus toxoid, a cellular or acellular pertussis antigen, and optionally one or more of conjugated H.influenzae type B capsular saccharide, hepatitis B virus surface antigen, and/or inactivated poliovirus antigens. For instance, the second immunogenic composition could be any of the products sold as PENTACEL™, PEDIACEL™, HEXAVAC, PEDIARIX™, INFANRIX PENTA™ INFANRIX HEXA™, QUINVAXEM™, EASYFIVE™ QUINTANRIX, TRITANRIX™, TRITANRIX-HEPB™, etc.

In one embodiment a subject receives (i) a first immunogenic composition comprising bacterial vesicles, (ii) an antipyretic, and (iii) a second immunogenic composition which is a pneumococcal conjugate vaccine. For instance, the second immunogenic composition could be any of the products sold as PREVNAR™, PREVNAR13™, SYNFLORIX™, etc.

In one embodiment a subject receives (i) a first immunogenic composition comprising bacterial vesicles, (ii) an antipyretic, and (iii) a second immunogenic composition which is a meningococcal conjugate vaccine. For instance, the second immunogenic composition could be any of the products sold as MENJUGATE™, MENINGITEC™, NEISVAC-C™, MENACTRA™, MENVEO™, MENITORIX™, NIMENRIX™, MENHIBRIX™, etc.

In one embodiment a subject receives (i) a first immunogenic composition comprising bacterial vesicles, (ii) an antipyretic, and (iii) a second immunogenic composition which is a rotavirus vaccine. For instance, the second immunogenic composition could be any of the products sold as ROTARIX™, ROTATEQ™, etc.

In one embodiment a subject receives (i) a first immunogenic composition comprising bacterial vesicles, (ii) an antipyretic, and (iii) a second immunogenic composition which is an influenza vaccine. For instance, the second immunogenic composition could be any of the products sold as AGRIPPAL™, BEGRIVAC™, FLUAD™, OPTAFLU™, FLUMIST™, FLUVIRIN™, INFLUVAC™, FLUZONE™, FLUARIX™, etc.

The first immunogenic composition, the antipyretic, and the second immunogenic composition should all be given within a single 24 hour period. The first and second immunogenic compositions will generally be given with 2 hours of each other, and they can be given in either order. They will usually be given by the same healthcare professional during a single visit to a healthcare centre.

In some embodiments, however, the subject does not receive a rotavirus vaccine i.e. neither at the same time as, or within 24 hours of, receiving the immunogenic composition comprising bacterial vesicles.

Similarly, in some embodiments, the subject does not receive a conjugated pneumococcal vaccine comprising a H.influenzae protein D carrier i.e. neither at the same time as, or within 24 hours of, receiving the immunogenic composition comprising bacterial vesicles. In other embodiments the subject does not receive a conjugated pneumococcal vaccine i.e. neither at the same time as, or within 24 hours of, receiving the immunogenic composition comprising bacterial vesicles.

### Meningococcal Antigens

The above text refers to various meningococcal antigens by name. Further details for some of these antigens are given below.

### NHBA (Neisserial Heparin Binding Antigen)

NHBA [37] was included in the published genome sequence for meningococcal serogroup B strain MC58 [57] as gene NMB2132 (GenBank accession number GI:7227388; SEQ ID NO: 9 herein). Sequences of NHBA from many strains have been published since then. For example, allelic forms of NHBA (referred to as protein ‘287’) can be seen in FIGS. 5 and 15 of reference 58, and in example 13 and FIG. 21 of reference 59 (SEQ IDs 3179 to 3184 therein). Various immunogenic fragments of NHBA have also been reported.

Preferred NHBA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 9; and/or (b) comprising a fragment of at least ‘n’ consecutive amino acids of SEQ ID NO: 9, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 9.

The most useful NHBA antigens can elicit antibodies which, after administration to a subject, can bind to a meningococcal polypeptide consisting of amino acid sequence SEQ ID NO: 9. Advantageous NHBA antigens for use with the invention can elicit bactericidal anti-meningococcal antibodies after administration to a subject.

### NadA (Neisserial Adhesin A)

The NadA antigen was included in the published genome sequence for meningococcal serogroup B strain MC58 [57] as gene NMB1994 (GenBank accession number GI:7227256; SEQ ID NO: 10 herein). The sequences of NadA antigen from many strains have been published since then, and the protein's activity as a Neisserial adhesin has been well documented. Various immunogenic fragments of NadA have also been reported.

Preferred NadA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 10; and/or (b) comprising a fragment of at least ‘n’ consecutive amino acids of SEQ ID NO: 10, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 10.

The most useful NadA antigens can elicit antibodies which, after administration to a subject, can bind to a meningococcal polypeptide consisting of amino acid sequence SEQ ID NO: 10. Advantageous NadA antigens for use with the invention can elicit bactericidal anti-meningococcal antibodies after administration to a subject. SEQ ID NO: 6 is one such fragment.

### HmbR

The full-length HmbR sequence was included in the published genome sequence for meningococcal serogroup B strain MC58 [57] as gene NMB1668 (SEQ ID NO: 7 herein). Reference 60 reports a HmbR sequence from a different strain (SEQ ID NO: 8 herein), and reference 41 reports a further sequence (SEQ ID NO: 19 herein). SEQ ID NOs: 7 and 8 differ in length by 1 amino acid and have 94.2% identity. SEQ ID NO: 19 is one amino acid shorter than SEQ ID NO: 7 and they have 99% identity (one insertion, seven differences) by CLUSTALW. The invention can use any such HmbR polypeptide.

The invention can use a polypeptide that comprises a full-length HmbR sequence, but it will often use a polypeptide that comprises a partial HmbR sequence. Thus in some embodiments a HmbR sequence used according to the invention may comprise an amino acid sequence having at least i % sequence identity to SEQ ID NO: 7, where the value of i is 50, 60, 70, 80, 90, 95, 99 or more. In other embodiments a HmbR sequence used according to the invention may comprise a fragment of at least j consecutive amino acids from SEQ ID NO: 7, where the value of j is 7, 8, 10, 12, 14, 16, 18, 20, 25, 30, 35, 40, 50, 60, 70, 80, 90, 100, 150, 200, 250 or more. In other embodiments a HmbR sequence used according to the invention may comprise an amino acid sequence (i) having at least i % sequence identity to SEQ ID NO: 7 and/or (ii) comprising a fragment of at least j consecutive amino acids from SEQ ID NO: 7.

Preferred fragments of j amino acids comprise an epitope from SEQ ID NO: 7. Such epitopes will usually comprise amino acids that are located on the surface of HmbR. Useful epitopes include those with amino acids involved in HmbR's binding to haemoglobin, as antibodies that bind to these epitopes can block the ability of a bacterium to bind to host haemoglobin. The topology of HmbR, and its critical functional residues, were investigated in reference 61. Fragments that retain a transmembrane sequence are useful, because they can be displayed on the bacterial surface e.g. in vesicles. Examples of long fragments of HmbR correspond to SEQ ID NOs: 21 and 22. If soluble HmbR is used, however, sequences omitting the transmembrane sequence, but typically retaining epitope(s) from the extracellular portion, can be used.

The most useful HmbR antigens can elicit antibodies which, after administration to a subject, can bind to a meningococcal polypeptide consisting of amino acid sequence SEQ ID NO: 7. Advantageous HmbR antigens for use with the invention can elicit bactericidal anti-meningococcal antibodies after administration to a subject.

fHbp (Factor H Binding Protein)

The fHbp antigen has been characterised in detail. It has also been known as protein ‘741’ [SEQ IDs 2535 & 2536 in ref. 59], ‘NMB1870’, ‘GNA1870’ [refs. 62-64], P2086′, ‘LP2086’ or ‘ORF2086’ [65-67]. It is naturally a lipoprotein and is expressed across all meningococcal serogroups. The structure of fHbp's C-terminal immunodominant domain (‘fHbpC’) has been determined by NMR [68]. This part of the protein forms an eight-stranded β-barrel, whose strands are connected by loops of variable lengths. The barrel is preceded by a short α-helix and by a flexible N-terminal tail.

The fHbp antigen falls into three distinct variants [69] and it has been found that serum raised against a given family is bactericidal within the same family, but is not active against strains which express one of the other two families i.e. there is intra-family cross-protection, but not inter-family cross-protection. The invention can use a single fHbp variant, but is will usefully include a fHbp from two or three of the variants. Thus it may use a combination of two or three different fHbps, selected from: (a) a first protein, comprising an amino acid sequence having at least a % sequence identity to SEQ ID NO: 1 and/or comprising an amino acid sequence consisting of a fragment of at least x contiguous amino acids from SEQ ID NO: 1; (b) a second protein, comprising an amino acid sequence having at least b % sequence identity to SEQ ID NO: 2 and/or comprising an amino acid sequence consisting of a fragment of at least y contiguous amino acids from SEQ ID NO: 2; and/or (c) a third protein, comprising an amino acid sequence having at least c % sequence identity to SEQ ID NO: 3 and/or comprising an amino acid sequence consisting of a fragment of at least z contiguous amino acids from SEQ ID NO: 3.

The value of a is at least 85 e.g. 86, 87, 88, 89, 90, 91, 92, 93, 94, 95, 96, 97, 98, 99, 99.5, or more. The value of b is at least 85 e.g. 86, 87, 88, 89, 90, 91, 92, 93, 94, 95, 96, 97, 98, 99, 99.5, or more. The value of c is at least 85 e.g. 86, 87, 88, 89, 90, 91, 92, 93, 94, 95, 96, 97, 98, 99, 99.5, or more. The values of a, b and c are not intrinsically related to each other.

The value of x is at least 7 e.g. 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 225, 250). The value of y is at least 7 e.g. 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 225, 250). The value of z is at least 7 e.g. 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 225, 250). The values of x, y and z are not intrinsically related to each other.

Where the invention uses a single fHbp variant, a composition may include a polypeptide comprising (a) an amino acid sequence having at least a % sequence identity to SEQ ID NO: 1 and/or comprising an amino acid sequence consisting of a fragment of at least x contiguous amino acids from SEQ ID NO: 1; or (b) an amino acid sequence having at least b % sequence identity to SEQ ID NO: 2 and/or comprising an amino acid sequence consisting of a fragment of at least y contiguous amino acids from SEQ ID NO: 2; or (c) an amino acid sequence having at least c % sequence identity to SEQ ID NO: 3 and/or comprising an amino acid sequence consisting of a fragment of at least z contiguous amino acids from SEQ ID NO: 3.

Where the invention uses a fHbp from two or three of the variants, a composition may include a combination of two or three different fHbps selected from: (a) a first polypeptide, comprising an amino acid sequence having at least a % sequence identity to SEQ ID NO: 1 and/or comprising an amino acid sequence consisting of a fragment of at least x contiguous amino acids from SEQ ID NO: 1; (b) a second polypeptide, comprising an amino acid sequence having at least b % sequence identity to SEQ ID NO: 2 and/or comprising an amino acid sequence consisting of a fragment of at least y contiguous amino acids from SEQ ID NO: 2; and/or (c) a third polypeptide, comprising an amino acid sequence having at least c % sequence identity to SEQ ID NO: 3 and/or comprising an amino acid sequence consisting of a fragment of at least z contiguous amino acids from SEQ ID NO: 3. The first, second and third polypeptides have different amino acid sequences.

Where the invention uses a fHbp from two of the variants, a composition can include both: (a) a first polypeptide, comprising an amino acid sequence having at least a % sequence identity to SEQ ID NO: 1 and/or comprising an amino acid sequence consisting of a fragment of at least x contiguous amino acids from SEQ ID NO: 1; and (b) a second polypeptide, comprising an amino acid sequence having at least b % sequence identity to SEQ ID NO: 2 and/or comprising an amino acid sequence consisting of a fragment of at least y contiguous amino acids from SEQ ID NO: 2. The first and second polypeptides have different amino acid sequences.

Where the invention uses a fHbp from two of the variants, a composition can include both: (a) a first polypeptide, comprising an amino acid sequence having at least a % sequence identity to SEQ ID NO: 1 and/or comprising an amino acid sequence consisting of a fragment of at least x contiguous amino acids from SEQ ID NO: 1; (b) a second polypeptide, comprising an amino acid sequence having at least c % sequence identity to SEQ ID NO: 3 and/or comprising an amino acid sequence consisting of a fragment of at least z contiguous amino acids from SEQ ID NO: 3. The first and second polypeptides have different amino acid sequences.

Another useful fHbp which can be used according to the invention is one of the modified forms disclosed, for example, in reference 70 e.g. comprising SEQ ID NO: 20 or 23 therefrom. These modified forms can elicit antibody responses which are broadly bactericidal against meningococci.

fHbp protein(s) in a OMV will usually be lipidated e.g. at a N-terminus cysteine. In other embodiments they will not be lipidated.

### NspA (Neisserial Surface Protein A)

The NspA antigen was included in the published genome sequence for meningococcal serogroup B strain MC58 [57] as gene NMB0663 (GenBank accession number GI:7225888; SEQ ID NO: 11 herein). The antigen was previously known from references 71 & 72. The sequences of NspA antigen from many strains have been published since then. Various immunogenic fragments of NspA have also been reported.

Preferred NspA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 11; and/or (b) comprising a fragment of at least ‘n’ consecutive amino acids of SEQ ID NO: 11, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 11.

The most useful NspA antigens can elicit antibodies which, after administration to a subject, can bind to a meningococcal polypeptide consisting of amino acid sequence SEQ ID NO: 11. Advantageous NspA antigens for use with the invention can elicit bactericidal anti-meningococcal antibodies after administration to a subject.

### NhhA (Neisseria Hia Homologue)

The NhhA antigen was included in the published genome sequence for meningococcal serogroup B strain MC58 [57] as gene NMB0992 (GenBank accession number GI:7226232; SEQ ID NO: 12 herein). The sequences of NhhA antigen from many strains have been published since e.g. refs 58 & 73, and various immunogenic fragments of NhhA have been reported. It is also known as Hsf.

Preferred NhhA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 12; and/or (b) comprising a fragment of at least ‘n’ consecutive amino acids of SEQ ID NO: 12, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 12.

The most useful NhhA antigens can elicit antibodies which, after administration to a subject, can bind to a meningococcal polypeptide consisting of amino acid sequence SEQ ID NO: 12. Advantageous NhhA antigens for use with the invention can elicit bactericidal anti-meningococcal antibodies after administration to a subject.

### App (Adhesion and Penetration Protein)

The App antigen was included in the published genome sequence for meningococcal serogroup B strain MC58 [57] as gene NMB1985 (GenBank accession number GI:7227246; SEQ ID NO: 13 herein). The sequences of App antigen from many strains have been published since then. It has also been known as ‘ORF1’ and ‘Hap’. Various immunogenic fragments of App have also been reported.

Preferred App antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 13; and/or (b) comprising a fragment of at least ‘n’ consecutive amino acids of SEQ ID NO: 13, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 13.

The most useful App antigens can elicit antibodies which, after administration to a subject, can bind to a meningococcal polypeptide consisting of amino acid sequence SEQ ID NO: 13. Advantageous App antigens for use with the invention can elicit bactericidal anti-meningococcal antibodies after administration to a subject.

### Omp85 (85 kDa Outer Membrane Protein)

The Omp85 antigen was included in the published genome sequence for meningococcal serogroup B strain MC58 [57] as gene NMB0182 (GenBank accession number GI:7225401; SEQ ID NO: 14 herein). The sequences of Omp85 antigen from many strains have been published since then. Further information on Omp85 can be found in references 74 and 75. Various immunogenic fragments of Omp85 have also been reported.

Preferred Omp85 antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 14; and/or (b) comprising a fragment of at least ‘n’ consecutive amino acids of SEQ ID NO: 14, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 14.

The most useful Omp85 antigens can elicit antibodies which, after administration to a subject, can bind to a meningococcal polypeptide consisting of amino acid sequence SEQ ID NO: 14. Advantageous Omp85 antigens for use with the invention can elicit bactericidal anti-meningococcal antibodies after administration to a subject.

### TbpA

The TbpA antigen was included in the published genome sequence for meningococcal serogroup B strain MC58 [57] as gene NMB0461 (GenBank accession number GI:7225687; SEQ ID NO: 23 herein). The sequences of TbpA from many strains have been published since then. Various immunogenic fragments of TbpA have also been reported.

Preferred TbpA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 23; and/or (b) comprising a fragment of at least ‘n’ consecutive amino acids of SEQ ID NO: 23, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 23.

The most useful TbpA antigens can elicit antibodies which, after administration to a subject, can bind to a meningococcal polypeptide consisting of amino acid sequence SEQ ID NO: 23. Advantageous TbpA antigens for use with the invention can elicit bactericidal anti-meningococcal antibodies after administration to a subject.

### TbpB

The TbpB antigen was included in the published genome sequence for meningococcal serogroup B strain MC58 [57] as gene NMB1398 (GenBank accession number GI:7225686; SEQ ID NO: 24 herein). The sequences of TbpB from many strains have been published since then. Various immunogenic fragments of TbpB have also been reported.

Preferred TbpB antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 24; and/or (b) comprising a fragment of at least ‘n’ consecutive amino acids of SEQ ID NO: 24, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 24.

The most useful TbpB antigens can elicit antibodies which, after administration to a subject, can bind to a meningococcal polypeptide consisting of amino acid sequence SEQ ID NO: 24. Advantageous TbpB antigens for use with the invention can elicit bactericidal anti-meningococcal antibodies after administration to a subject.

### Cu,Zn-Superoxide Dismutase

The Cu,Zn-superoxide dismutase antigen was included in the published genome sequence for meningococcal serogroup B strain MC58 [57] as gene NMB1398 (GenBank accession number GI:7226637; SEQ ID NO: 25 herein). The sequences of Cu,Zn-superoxide dismutase from many strains have been published since then. Various immunogenic fragments of Cu,Zn-superoxide dismutase have also been reported.

Preferred Cu,Zn-superoxide dismutase antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 25; and/or (b) comprising a fragment of at least ‘n’ consecutive amino acids of SEQ ID NO: 25, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 25.

The most useful Cu,Zn-superoxide dismutase antigens can elicit antibodies which, after administration to a subject, can bind to a meningococcal polypeptide consisting of amino acid sequence SEQ ID NO: 25. Advantageous Cu,Zn-superoxide dismutase antigens for use with the invention can elicit bactericidal anti-meningococcal antibodies after administration to a subject.

### Other Meningococcal Immunogenic Compositions

The invention is discussed above by reference to immunogenic compositions which comprise bacterial vesicles. In alternative embodiments an immunogenic composition used with the invention does not comprise bacterial vesicles but does comprise one or more of: (i) a meningococcal fHbp antigen; (ii) a meningococcal NHBA antigen; and/or (iii) a meningococcal NadA antigen.

The invention is particularly useful with an immunogenic composition which does not comprise bacterial vesicles but does comprise a meningococcal fHbp antigen.

### General

The practice of the present invention will employ, unless otherwise indicated, conventional methods of chemistry, biochemistry, molecular biology, immunology and pharmacology, within the skill of the art. Such techniques are explained fully in the literature. See, e.g., references 76-82, etc.

The term “comprising” encompasses “including” as well as “consisting” e.g. a composition “comprising” X may consist exclusively of X or may include something additional e.g. X+Y.

The term “about” in relation to a numerical value x is optional and means, for example, x+10%.

Where the invention concerns an “epitope”, this epitope may be a B-cell epitope and/or a T-cell epitope, but will usually be a B-cell epitope. Such epitopes can be identified empirically (e.g. using PEPSCAN [83,84] or similar methods), or they can be predicted (e.g. using the Jameson-Wolf antigenic index [85], matrix-based approaches [86], MAPITOPE [87], TEPITOPE [88,89], neural networks [90], OptiMer & EpiMer [91, 92], ADEPT [93], Tsites [94], hydrophilicity [95], antigenic index [96] or the methods disclosed in references 97-101, etc.). Epitopes are the parts of an antigen that are recognised by and bind to the antigen binding sites of antibodies or T-cell receptors, and they may also be referred to as “antigenic determinants”.

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of ref. 102. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in ref. 103.

The word “substantially” does not exclude “completely” e.g. a composition which is “substantially free” from Y may be completely free from Y. Where necessary, the word “substantially” may be omitted from the definition of the invention.

### MODES FOR CARRYING OUT THE INVENTION

Infants aged approximately 2 months are enrolled into a clinical trial. Three groups are immunised with INFANRIX HEXA™ and PREVENAR™ plus:
I: the OMV-containing BEXSERO™ vaccine as described in reference 56.
II: as group I, but with concomitant prophylactic administration of paracetamol.
III: the MENJUGATE™ meningococcal conjugate.

These treatments are given at 2, 3, and 4 months of age i.e. with a 3-dose regimen. In group II the subjects are given one dose of paracetamol just before vaccination. Parents are instructed to administer two further doses at 4-6 hour intervals after vaccination. Paracetamol is administered orally, at the dose of 10-15 mg/kg, If additional doses of paracetamol are administered therapeutically for post-vaccination reactions, no more than 4 total doses are given over 24 hours.

Body temperature is measured after each injection (to assess fever), and blood is taken at 5 months of age (to assess immunogenicity). The proportion of patients with elevated body temperatures after each injection is as follows:

| | I | | II | | III | |
|---|---|---|---|---|---|---|
| | >38.5° | >39.5° | >38.5° | >39.5° | >38.5° | >39.5° |
| | C. | C. | C. | C. | C. | C. |
| 1 | 51% | 39% | 25% | 1% | 12% | 0% |
| 2 | 49% | 4% | 19% | 1% | 17% | 1% |
| 3 | 30% | 3% | 11% | 1% | 8% | 1% |

A serum bactericidal assay is performed using blood taken at 5 months. Titers against the main meningococcal vaccine antigens (fHbp, NadA, NHBA) and against a control meningococcal antigen (PorA) are as follows:

| | fHbp | NadA | NHBA | PorA |
|---|---|---|---|---|
| I | 100 | 394 | 5.2 | 9.9 |
| II | 100 | 451 | — | 8.45 |
| III | 1.3 | 1.2 | 1.0 | 1.1 |

The proportion of subjects with an increase in SBA titer of ≧1:5 is as follows:

| | fHbp | NadA | NHBA | PorA |
|---|---|---|---|---|
| I | 100% | 99% | 43% | 76% |
| II | 100% | 99% | — | 75% |
| III | 6% | 3% | 20% | 2% |

At 5 months, the proportion of seroresponders (≧0.35 μg/ml) against the 7 pneumococcal serotypes in the PREVENAR™ vaccine is as follows:

| | 4 | 6B | 9V | 14 | 18C | 19F | 23F |
|---|---|---|---|---|---|---|---|
| I | 95% | 76% | 100% | 98% | 99% | 99% | 95% |
| II | 91% | 73% | 99% | 93% | 98% | 98% | 92% |

Immunogenicity of the INFANRIX HEXA™ antigens is as follows:

| | D ≧0.1 | T ≧0.1 | | | | | | | HBV ≧10 | Hib ≧0.15 |
|---|---|---|---|---|---|---|---|---|---|---|
| | IU/ml | IU/ml | Prn | PT | FHA | IPV1 ≧1:8 | IPV2 ≧1:8 | IPV3 ≧1:8 | mIU/mL | μg/mL |
| I | 100% | 100% | 97% | 98% | 97% | 99% | 96% | 100% | 97% | 98% |
| II | 100% | 100% | 91% | 97% | 97% | 97% | 96% | 100% | 97% | 99% |

Thus the prophylactic paracetamol treatment has a significant effect (reduction of ˜50%) on fever rates but does not negatively affect the immunogenicity of either the meningococcal vaccine or the routine non-meningococcal vaccines. These findings contrast with references 2 and 4.

It will be understood that the invention is described above by way of example only and modifications may be made whilst remaining within the scope and spirit of the invention.

### REFERENCES

[1] Nøkleby et al. (2007) Vaccine 25:3080-4.
[2] Prymula et al. (2009) Lancet 374:1339-50.
[3] Chen et al. (2009) Lancet 374:1305-6.
[4] Prymula et al. (2011) Vaccine 29:1959-67.
[5] Jackson et al. (2006) Pediatrics 117:620-5.
[6] Manley et al. (2007) Annals Pharmacotherapy 41:1227-32.
[7] Kratz et al. (2004) NEJM 351:1548-63.
[8] WO02/09643.
[9] Katial et al. (2002) Infect. Immun. 70:702-707.
[10] U.S. Pat. No. 6,180,111.
[11] WO01/34642.
[12] WO2004/019977.
[13] European patent 0011243.
[14] Fredriksen et al. (1991) NIPH Ann. 14(2):67-80.
[15] WO01/91788.
[16] WO2005/004908.
[17] WO2006/046143.
[18] Claassen et al. (1996) Vaccine 14:1001-8.
[19] Koeberling et al. (2007) Vaccine 25:1912-20.
[20] Koeberling et al. (2008) J Infect Dis 198:262-70.
[21] Hou et al. (2005) Jlnfect Dis 192:580-90.
[22] WO2009/038889.
[23] Bonvehi et al. (2010) Clin Vacc Immunol 17:1460-6.
[24] Zollinger et al. (2010) Vaccine 28:5057-67.
[25] WO99/10497.
[26] Steeghs et al. (2001) The EMBO Journal 20:6937-6945.
[27] Maiden et al. (1998) PNAS USA 95:3140-3145.
[28] WO01/09350.
[29] WO02/062378.
[30] WO2004/014417.
[31] WO00/26384.
[32] U.S. Pat. No. 6,531,131
[33] U.S. Pat. No. 6,645,503
[34] WO2004/015099.
[35] WO2006/081259.
[36] Deghmane et al. (2003) Infect Immun 71:2897-901.
[37] Serruto et al. (2010) PNAS USA 107:3770-5.
[38] Schielke et al. (2009) Mol Microbiol 72:1054-67.
[39] WO2004/014418.
[40] WO00/25811.
[41] WO2010/070453.
[42] WO02/09746.
[43] Oriente et al. (2010) J Bacteriol 192:691-701.
[44] U.S. provisional patent application 61/247,428.
[45] Giuliani et al. (2006) Proc Natl Acad Sci USA 103(29):10834-9.
[46] Donnelly et al. (2010) PNAS USA 107:19490-5.
[47] Kimura et al. (2010) Clin Vaccine Immunol. 2010 PMID: 21177912.
[48] de Kleijn et al. (2000) Vaccine 18:1456-66.
[49] WO03/105890.
[50] WO2006/024946
[51] WO01/30390.
[52] Gennaro (2000) Remington: The Science and Practice of Pharmacy. 20th edition, ISBN: 0683306472.
[53] RIVM report 000012 003.
[54] Vaccine Design . . . (1995) eds. Powell & Newman. ISBN: 030644867X. Plenum.
[55] RIVM report 124001 004.
[56] Findlow et al. (2010) Clin Infect Dis 51:1127-37.
[57] Tettelin et al. (2000) Science 287:1809-1815.
[58] WO00/66741.
[59] WO99/57280
[60] U.S. Pat. No. 5,698,438.
[61] Perkins-Balding et al. (2003) Microbiology 149:3423-35.
[62] Masignani et al. (2003) J Exp Med 197:789-799.
[63] Welsch et al. (2004) J Immunol 172:5605-15.
[64] Hou et al. (2005) J Infect Dis 192(4):580-90.
[65] WO03/063766.
[66] Fletcher et al. (2004) Infect Immun 72:2088-2100.
[67] Zhu et al. (2005) Infect Immun 73(10):6838-45.
[68] Cantini et al. (2006) J. Biol. Chem. 281:7220-7227
[69] WO2004/048404
[70] WO2009/104097.
[71] Martin et al. (1997) J Exp Med 185(7):1173-83.
[72] WO96/29412.
[73] WO01/55182.
[74] WO01/38350.
[75] WO00/23595.
[76] Methods In Enzymology (S. Colowick and N. Kaplan, eds., Academic Press, Inc.)
[77] Handbook of Experimental Immunology, Vols. I-IV (D. M. Weir and C.C. Blackwell, eds, 1986, Blackwell Scientific Publications)
[78] Sambrook et al. (2001) Molecular Cloning: A Laboratory Manual, 3rd edition (Cold Spring Harbor Laboratory Press).
[79] Handbook of Surface and Colloidal Chemistry (Birdi, K. S. ed., CRC Press, 1997)
[80] Ausubel et al. (eds) (2002) Short protocols in molecular biology, 5th edition (Current Protocols).
[81] Molecular Biology Techniques: An Intensive Laboratory Course, (Ream et al., eds., 1998, Academic Press)
[82] PCR (Introduction to Biotechniques Series), 2nd ed. (Newton & Graham eds., 1997, Springer Verlag)
[83] Geysen et al. (1984) PNAS USA 81:3998-4002.
[84] Carter (1994) Methods Mol Biol 36:207-23.
[85] Jameson, B A et al. 1988, CABIOS 4(1):181-186.
[86] Raddrizzani & Hammer (2000) Brief Bioinform 1(2):179-89.
[87] Bublil et al. (2007) Proteins 68(1):294-304.
[88] De Lalla et al. (1999) J. Immunol. 163:1725-29.
[89] Kwok et al. (2001) Trends Immunol 22:583-88.
[90] Brusic et al. (1998) Bioinformatics 14(2):121-30
[91] Meister et al. (1995) Vaccine 13(6):581-91.
[92] Roberts et al. (1996) AIDS Res Hum Retroviruses 12(7):593-610.
[93] Maksyutov & Zagrebelnaya (1993) Comput Appl Biosci 9(3):291-7.
[94] Feller & de la Cruz (1991) Nature 349(6311):720-1.
[95] Hopp (1993) Peptide Research 6:183-190.
[96] Welling et al. (1985) FEBS Lett. 188:215-218.
[97] Davenport et al. (1995) Immunogenetics 42:392-297.
[98] Tsurui & Takahashi (2007) J Pharmacol Sci. 105(4):299-316.
[99] Tong et al. (2007) Brief Bioinform. 8(2):96-108.
[100] Schirle et al. (2001) J Immunol Methods. 257(1-2):1-16.
[101] Chen et al. (2007) Amino Acids 33(3):423-8.
[102] Current Protocols in Molecular Biology (F. M. Ausubel et al., eds., 1987) Supplement 30
[103] Smith & Waterman (1981) Adv. Appl. Math. 2: 482-489.

1. A method for immunising a human subject, wherein the subject receives (i) an immunogenic composition comprising bacterial vesicles and (ii) an antipyretic, and wherein the immunogenic composition and the antipyretic are administered to the subject within 24 hours of each other.

2. An antipyretic and an immunogenic composition comprising bacterial vesicles, for combined use in a method of immunising a human subject, wherein the immunogenic composition and the antipyretic are administered to the subject within 24 hours of each other.

3. In a method for immunising a human subject by administering an immunogenic composition comprising bacterial vesicles, an improvement consisting of administering an antipyretic to the subject within 24 hours of administering the immunogenic composition.

4. A combination of (i) an antipyretic and (ii) an immunogenic composition comprising bacterial vesicles, for simultaneous, separate or sequential administration, wherein components (i) and (ii) are administered within 24 hours of each other.

5. A kit comprising (i) an antipyretic and (ii) an immunogenic composition comprising bacterial vesicles.

6. A package comprising (i) an immunogenic composition comprising bacterial vesicles and (ii) an information leaflet (a) containing written instructions that an antipyretic may be administered to a subject within 24 hours of their receiving the immunogenic composition and/or (b) instructing a subject or physician to administer an antipyretic to the subject if the subject develops a fever after receiving the immunogenic composition.

7. The method, composition, improvement, combination, kit or package of any preceding claim, wherein the antipyretic is administered (i) no more than 2 hours before the immunogenic composition (ii) at the same time as the immunogenic composition or (iii) no more than 2 hours after the immunogenic composition.

8. The method, composition, improvement, combination, kit or package of any preceding claim, wherein the subject is less than 1 year old.

9. The method, composition, improvement, combination, kit or package of any preceding claim, wherein the vesicles are meningococcal outer membrane vesicles.

10. The method, composition, improvement, combination, kit or package of claim 9, wherein the vesicles are prepared from a serogroup B meningococcus.

11. The method, composition, improvement, combination, kit or package of claim 10, wherein the immunogenic composition includes a protein comprising SEQ ID NO: 4, a protein comprising SEQ ID NO: 5, and a protein comprising SEQ ID NO: 6.

12. The method, composition, improvement, combination, kit or package of claim 10, wherein the vesicles are prepared from a meningococcus in which TbpA expression is upregulated.

13. The method, composition, improvement, combination, kit or package of claim 10 or claim 12, wherein the vesicles are prepared from a meningococcus in which NhhA expression is upregulated.

14. The method, composition, improvement, combination, kit or package of claim 10 or claim 12 or claim 13, wherein the vesicles are prepared from a meningococcus in which fHbp expression is upregulated.

15. The method, composition, improvement, combination, kit or package of claim 10 or claim 12 or claim 13 or claim 14, wherein the vesicles are prepared from a meningococcus in which PorA expression is downregulated.

16. A method for immunising a human subject, wherein the subject receives (i) an immunogenic composition comprising a meningococcal fHbp antigen and (ii) an antipyretic, and wherein the immunogenic composition and the antipyretic are administered to the subject within 24 hours of each other.

17. An antipyretic and an immunogenic composition comprising a meningococcal fHbp antigen, for combined use in a method of immunising a human subject, wherein the immunogenic composition and the antipyretic are administered to the subject within 24 hours of each other.

18. In a method for immunising a human subject by administering an immunogenic composition comprising a meningococcal fHbp antigen, an improvement consisting of administering an antipyretic to the subject within 24 hours of administering the immunogenic composition.

19. A combination of (i) an antipyretic and (ii) an immunogenic composition comprising a meningococcal fHbp antigen, for simultaneous, separate or sequential administration, wherein components (i) and (ii) are administered within 24 hours of each other.

20. A kit comprising (i) an antipyretic and (ii) an immunogenic composition comprising a meningococcal fHbp antigen.

21. A package comprising (i) an immunogenic composition comprising a meningococcal fHbp antigen and (ii) an information leaflet (a) containing written instructions that an antipyretic may be administered to a subject within 24 hours of their receiving the immunogenic composition and/or (b) instructing a subject or physician to administer an antipyretic to the subject if the subject develops a fever after receiving the immunogenic composition.

22. The method, composition, improvement, combination, kit or package of any one of claims 16 to 21, wherein the antipyretic is administered (i) no more than 2 hours before the immunogenic composition (ii) at the same time as the immunogenic composition or (iii) no more than 2 hours after the immunogenic composition.

23. The method, composition, improvement, combination, kit or package of any one of claims 16 to 22, wherein the subject is less than 1 year old.

24. The method, composition, improvement, combination, kit or package of any one of claims 16 to 23, wherein the immunogenic composition includes a protein comprising SEQ ID NO: 5.

25. The method, composition, improvement, combination, kit or package of any preceding claim, wherein the combination of an antipyretic and an immunogenic composition is administered to a subject 2 or more times.

26. The method, composition, improvement, combination, kit or package of claim 25, wherein the combination of an antipyretic and an immunogenic composition is administered to a subject 3 times.

27. The method, composition, improvement, combination, kit or package of any one of claims 25 to 26, wherein each administration of the combination in a series is administered within 1 or 2 months of the preceding administration of the combination in the series.

28. The method, composition, improvement, combination, kit or package of claims 25 to 27, wherein the combination is first administered to a subject at 2 months of age, followed by a second administration of the combination at 3 months of age, and a third administration of the combination at 4 months of age.

29. The method, composition, improvement, combination, kit or package of any preceding claim, wherein the immunogenic composition includes an aluminium salt adjuvant.

30. The method, composition, improvement, combination, kit or package of any preceding claim, wherein the immunogenic composition is administered by intramuscular injection.

31. The method, composition, improvement, combination, kit or package of any preceding claim, wherein the antipyretic is acetaminophen.

32. The method, composition, improvement, combination, kit or package of any preceding claim, wherein the antipyretic is administered before the immunogenic composition.

33. The method, composition, improvement, combination, kit or package of any preceding claim, wherein the immunogenic composition and the antipyretic are administered within 1 hour of each other.

34. The method, composition, improvement, combination, kit or package of any preceding claim, wherein the subject receives the immunogenic composition once and the antipyretic at least twice within a 24 hour period.

35. The method, composition, improvement, combination, kit or package of claim 34, wherein antipyretic is administered both before and after the immunogenic composition.

36. The method, composition, improvement, combination, kit or package of any preceding claim, wherein the antipyretic will be administered orally.

37. The method, composition, improvement, combination, kit or package of any preceding claim, wherein the subject also receives an immunogenic composition which does not comprise bacterial vesicles.


SEQUENCE LISTING (25 sequences)

SEQ ID NO: 1 - protein, 248 aa, Neisseria meningitidis
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 2 - protein, 247 aa, Neisseria meningitidis
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 3 - protein, 250 aa, Neisseria meningitidis
VAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 4 - protein, 644 aa, Artificial Sequence
  Hybrid meningococcal antigen
MASPDVKSADTLSKPAAPVVSEKETEAKEDAPQAGSQGQGAPSAQGGQDMAAVSEENTGNGGAAATDKPKNEDEGAQNDMPQNAADTDSLTPNHTPASNMPAGNMENQAPDAGESEQPANQPDMANTADGMQGDDPSAGGENAGNTAAQGTNQAENNQTAGSQNPASSTNPSATNSGGDFGRTNVGNSVVIDGPSQNITLTHCKGDSCSGNNFLDEEVQLKSEFEKLSDADKISNYKKDGKNDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPSKGEMLAGTAVYNGEVLHFHTENGRPSPSRGRFAAKVDFGSKSVDGIIDSGDGLHMGTQKFKAAIDGNGFKGTWTENGGGDVSGKFYGPAGEEVAGKYSYRPTDAEKGGFGVFAGKKEQDGSGGGGATYKVDEYHANARFAIDHFNTSTNVGGFYGLTGSVEFDQAKRDGKIDITIPVANLQSGSQHFTDHLKSADIFDAAQYPDIRFVSTKFNFNGKKLVSVDGNLTMHGKTAPVKLKAEKFNCYQSPMAKTEVCGGDFSTTIDRTKWGVDYLVNVGMTKSVRIDIQIEAAKQ

SEQ ID NO: 5 - protein, 434 aa, Artificial Sequence
  Hybrid meningococcal antigen
MVSAVIGSAAVGAKSAVDRRTTGAQTDDNVMALRIETTARSYLRQNNQTKGYTPQISVVGYDRHLLLLGQVATEGEKQFVGQIARSEQAAEGVYNYITVASLPRTAGDIAGDTWNTSKVRATLLGISPATRARVKIVTYGNVTYVMGILTPEEQAQITQKVSTTVGVQKVITLYQNYVQRGSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 6 - protein, 327 aa, Neisseria meningitidis
ATNDDDVKKAATVAIAAAYNNGQEINGFKAGETIYDIDEDGTITKKDATAADVEADDFKGLGLKKVVTNLTKTVNENKQNVDAKVKAAESEIEKLTTKLADTDAALADTDAALDATTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKDNIAKKANSADVYTREESDSKFVRIDGLNATTEKLDTRLASAEKSIADHDTRLNGLDKTVSDLRKETRQGLAEQAALSGLFQPYNVG

SEQ ID NO: 7 - protein, 792 aa, Neisseria meningitidis
MKPLQMLPIAALVGSIFGNPVLAADEAATETTPVKAEIKAVRVKGQRNAPAAVERVNLNRIKQEMIRDNKDLVRYSTDVGLSDSGRHQKGFAVRGVEGNRVGVSIDGVNLPDSEENSLYARYGNFNSSRLSIDPELVRNIEIVKGADSFNTGSGALGGGVNYQTLQGRDLLLDDRQFGVMMKNGYSTRNREWTNTLGFGVSNDRVDAALLYSQRRGHETESAGNRGYAVEGEGSGANIRGSARGIPDSSKHKYNHHALGKIAYQINDNHRIGASLNGQQGHNYTVEESYNLTASSWREADDVNRRRNANLFYEWMPDSNWLSSLKADFDYQKTKVAAVNNKGSFPMDYSTWTRNYNQKDLDEIYNRSMDTRFKRFTLRLDSHPLQLGGGRHRLSFKTFVSRRDFENLNRDDYYFSGRVVRTTSSIQHPVKTTNYGFSLSDQIQWNDVFSSRAGIRYDHTKMTPQELNAECHACDKTPPAANTYKGWSGFVGLAAQLNQAWRVGYDITSGYRVPNASEVYFTYNHGSGNWLPNPNLKAERSTTHTLSLQGRSEKGMLDANLYQSNYRNFLSEEQKLTTSGTPGCTEENAYYGICSDPYKEKLDWQMKNIDKARIRGIELTGRLNVDKVASFVPEGWKLFGSLGYAKSKLSGDNSLLSTQPLKVIAGIDYESPSEKWGVFSRLTYLGAKKVKDAQYTVYENKGWGTPLQKKVKDYPWLNKSAYVFDMYGFYKPAKNLTLRAGVYNLFNRKYTTWDSLRGLYSYSTTNAVDRDGKGLDRYRAPGRNYAVSLEWKF

SEQ ID NO: 8 - protein, 793 aa, Neisseria meningitidis
MKPLQMLPIAALVGSIFGNPVFAADEAATETTPVKAEVKAVRVKGQRNAPAAVERVNLNRIKQEMIRDNKDLVRYSTDVGLSDSGRHQKGFAVRGVEGNRVGVSIDGVNLPDSEENSLYARYGNFNSSRLSIDPELVRNIDIVKGADSFNTGSGALGGGVNYQTLQGRDLLLPERQFGVMMKNGYSTRNREWTNTLGFGVSNDRVDAALLYSQRRGHETESAGKRGYPVEGAGSGANIRGSARGIPDPSQHKYNHHALGKIAYQINDNHRIGASLNGQQGHNYTVEESYNLLASYWREADDVNRRRNTNLFYEWTPESDRLSMVKADVDYQKTKVSAVNYKGSFPIEDSSTLTRNYNQKDLDEIYNRSMDTRFKRITLRLDSHPLQLGGGRHRLSFKTFASRRDFENLNRDDYYFSGRVVRTTSSIQHPVKTTNYGFSLSDQIQWNDVFSSRAGIRYDHTKMTPQELNAECHACDKTPPAANTYKGWSGFVGLAAQLNQAWRVGYDITSGYRVPNASEVYFTYNHGSGNWLPNPNLKAERTTTHTLSLQGRSEKGTLDANLYQSNYRNFLSEEQKLTTSGDVSCTQMNYYYGMCSNPYSEKLEWQMQNIDKARIRGIELTGRLNVDKVASFVPEGWKLFGSLGYAKSKLSGDNSLLSTQPLKVIAGIDYESPSEKWGVFSRLTYLGAKKVKDAQYTVYENKGWGTPLQKKVKDYPWLNKSAYVFDMYGFYKPVKNLTLRAGVYNVFNRKYTTWDSLRGLYSYSTTNSVDRDGKGLDRYRAPSRNYAVSLEWKF

SEQ ID NO: 9 - protein, 488 aa, Neisseria meningitidis
MFKRSVIAMACIFALSACGGGGGGSPDVKSADTLSKPAAPVVSEKETEAKEDAPQAGSQGQGAPSAQGSQDMAAVSEENTGNGGAVTADNPKNEDEVAQNDMPQNAAGTDSSTPNHTPDPNMLAGNMENQATDAGESSQPANQPDMANAADGMQGDDPSAGGQNAGNTAAQGANQAGNNQAAGSSDPIPASNPAPANGGSNFGRVDLANGVLIDGPSQNITLTHCKGDSCSGNNFLDEEVQLKSEFEKLSDADKISNYKKDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPAKGEMLAGAAVYNGEVLHFHTENGRPYPTRGRFAAKVDFGSKSVDGIIDSGDDLHMGTQKFKAAIDGNGFKGTWTENGSGDVSGKFYGPAGEEVAGKYSYRPTDAEKGGFGVFAGKKEQD

SEQ ID NO: 10 - protein, 364 aa, Neisseria meningitidis
MSMKHFPSKVLTTAILATFCSGALAATSDDDVKKAATVAIVAAYNNGQEINGFKAGETIYDIGEDGTITQKDATAADVEADDFKGLGLKKVVTNLTKTVNENKQNVDAKVKAAESEIEKLTTKLADTDAALADTDAALDETTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKADIAKNSARIDSLDKNVANLRKETRQGLAEQAALSGLFQPYNVGRFNVTAAVGGYKSESAVAIGTGFRFTENFAAKAGVAVGTSSGSSAAYHVGVNYEW

SEQ ID NO: 11 - protein, 174 aa, Neisseria meningitidis
MKKALATLIALALPAAALAEGASGFYVQADAAHAKASSSLGSAKGFSPRISAGYRINDLRFAVDYTRYKNYKAPSTDFKLYSIGASAIYDFDTQSPVKPYLGARLSLNRASVDLGGSDSFSQTSIGLGVLTGVSYAVTPNVDLDAGYRYNYIGKVNTVKNVRSGELSAGVRVKF

SEQ ID NO: 12 - protein, 591 aa, Neisseria meningitidis
MNKIYRIIWNSALNAWVVVSELTRNHTKRASATVKTAVLATLLFATVQASANNEEQEEDLYLDPVQRTVAVLIVNSDKEGTGEKEKVEENSDWAVYFNEKGVLTAREITLKAGDNLKIKQNGTNFTYSLKKDLTDLTSVGTEKLSFSANGNKVNITSDTKGLNFAKETAGTNGDTTVHLNGIGSTLTDTLLNTGATTNVTNDNVTDDEKKRAASVKDVLNAGWNIKGVKPGTTASDNVDFVRTYDTVEFLSADTKTTTVNVESKDNGKKTEVKIGAKTSVIKEKDGKLVTGKDKGENGSSTDEGEGLVTAKEVIDAVNKAGWRMKTTTANGQTGQADKFETVTSGTNVTFASGKGTTATVSKDDQGNITVMYDVNVGDALNVNQLQNSGWNLDSKAVAGSSGKVISGNVSPSKGKMDETVNINAGNNIEITRNGKNIDIATSMTPQFSSVSLGAGADAPTLSVDGDALNVGSKKDNKPVRITNVAPGVKEGDVTNVAQLKGVAQNLNNRIDNVDGNARAGIAQAIATAGLVQAYLPGKSMMAIGGGTYRGEAGYAIGYSSISDGGNWIIKGTASGNSRGHFGASASVGYQW

SEQ ID NO: 13 - protein, 1457 aa, Neisseria meningitidis
MKTTDKRTTETHRKAPKTGRIRFSPAYLAICLSFGILPQAWAGHTYFGINYQYYRDFAENKGKFAVGAKDIEVYNKKGELVGKSMTKAPMIDFSVVSRNGVAALVGDQYIVSVAHNGGYNNVDFGAEGRNPDQHRFTYKIVKRNNYKAGTKGHPYGGDYHMPRLHKFVTDAEPVEMTSYMDGRKYIDQNNYPDRVRIGAGRQYWRSDEDEPNNRESSYHIASAYSWLVGGNTFAQNGSGGGTVNLGSEKIKHSPYGFLPTGGSFGDSGSPMFIYDAQKQKWLINGVLQTGNPYIGKSNGFQLVRKDWFYDEIFAGDTHSVFYEPRQNGKYSFNDDNNGTGKINAKHEHNSLPNRLKTRTVQLFNVSLSETAREPVYHAAGGVNSYRPRLNNGENISFIDEGKGELILTSNINQGAGGLYFQGDFTVSPENNETWQGAGVHISEDSTVTWKVNGVANDRLSKIGKGTLHVQAKGENQGSISVGDGTVILDQQADDKGKKQAFSEIGLVSGRGTVQLNADNQFNPDKLYFGFRGGRLDLNGHSLSFHRIQNTDEGAMIVNHNQDKESTVTITGNKDIATTGNNNSLDSKKEIAYNGWFGEKDTTKTNGRLNLVYQPAAEDRTLLLSGGTNLNGNITQTNGKLFFSGRPTPHAYNHLNDHWSQKEGIPRGEIVWDNDWINRTFKAENFQIKGGQAVVSRNVAKVKGDWHLSNHAQAVFGVAPHQSHTICTRSDWTGLTNCVEKTITDDKVIASLTKTDISGNVDLADHAHLNLTGLATLNGNLSANGDTRYTVSHNATQNGNLSLVGNAQATFNQATLNGNTSASGNASFNLSDHAVQNGSLTLSGNAKANVSHSALNGNVSLADKAVFHFESSRFTGQISGGKDTALHLKDSEWTLPSGTELGNLNLDNATITLNSAYRHDAAGAQTGSATDAPRRRSRRSRRSLLSVTPPTSVESRFNTLTVNGKLNGQGTFRFMSELFGYRSDKLKLAESSEGTYTLAVNNTGNEPASLEQLTVVEGKDNKPLSENLNFTLQNEHVDAGAWRYQLIRKDGEFRLHNPVKEQELSDKLGKAEAKKQAEKDNAQSLDALIAAGRDAVEKTESVAEPARQAGGENVGIMQAEEEKKRVQADKDTALAKQREAETRPATTAFPRARRARRDLPQLQPQPQPQPQRDLISRYANSGLSEFSATLNSVFAVQDELDRVFAEDRRNAVWTSGIRDTKHYRSQDFRAYRQQTDLRQIGMQKNLGSGRVGILFSHNRTENTFDDGIGNSARLAHGAVFGQYGIDRFYIGISAGAGFSSGSLSDGIGGKIRRRVLHYGIQARYRAGFGGFGIEPHIGATRYFVQKADYRYENVNIATPGLAFNRYRAGIKADYSFKPAQHISITPYLSLSYTDAASGKVRTRVNTAVLAQDFGKTRSAEWGVNAEIKGFTLSLHAAAAKGPQLEAQHSAGIKLGYRW

SEQ ID NO: 14 - protein, 797 aa, Neisseria meningitidis
MKLKQIASALMMLGISPLALADFTIQDIRVEGLQRTEPSTVFNYLPVKVGDTYNDTHGSAIIKSLYATGFFDDVRVETADGQLLLTVIERPTIGSLNITGAKMLQNDAIKKNLESFGLAQSQYFNQATLNQAVAGLKEEYLGRGKLNIQITPKVTKLARNRVDIDITIDEGKSAKITDIEFEGNQVYSDRKLMRQMSLTEGGIWTWLTRSNQFNEQKFAQDMEKVTDFYQNNGYFDFRILDTDIQTNEDKTKQTIKITVHEGGRFRWGKVSIEGDTNEVPKAELEKLLTMKPGKWYERQQMTAVLGEIQNRMGSAGYAYSEISVQPLPNAETKTVDFVLHIEPGRKIYVNEIHITGNNKTRDEVVRRELRQMESAPYDTSKLQRSKERVELLGYFDNVQFDAVPLAGTPDKVDLNMSLTERSTGSLDLSAGWVQDTGLVMSAGVSQDNLFGTGKSAALRASRSKTTLNGSLSFTDPYFTADGVSLGYDVYGKAFDPRKASTSIKQYKTTTAGAGIRMSVPVTEYDRVNFGLVAEHLTVNTYNKAPKHYADFIKKYGKTDGTDGSFKGWLYKGTVGWGRNKTDSALWPTRGYLTGVNAEIALPGSKLQYYSATHNQTWFFPLSKTFTLMLGGEVGIAGGYGRTKEIPFFENFYGGGLGSVRGYESGTLGPKVYDEYGEKISYGGNKKANVSAELLFPMPGAKDARTVRLSLFADAGSVWDGKTYDDNSSSATGGRVQNIYGAGNTHKSTFTNELRYSAGGAVTWLSPLGPMKFSYAYPLKKKPEDEIQRFQFQLGTTF

SEQ ID NO: 15 - protein, 6 aa, Artificial Sequence
  Linker
GSGGGG

SEQ ID NO: 16 - protein, 8 aa, Artificial Sequence
  Linker
GSGSGGGG

SEQ ID NO: 17 - protein, 6 aa, Artificial Sequence
  Poly-histidine tag
HHHHHH

SEQ ID NO: 18 - DNA, 2376 bp, Neisseria meningitidis
atgaaaccattacaaatgctccctatcgccgcgctggtcggcagtattttcggcaatccggtcttggcagcagatgaagctgcaactgaaaccacacccgttaaggcagagataaaagcagtgcgcgttaaaggtcagcgcaatgcgcctgcggctgtggaacgcgtcaaccttaaccgtatcaaacaagaaatgatacgcgacaataaagacttggtgcgctattccaccgatgtcggcttgagcgacagcggccgccatcaaaaaggctttgctgttcgcggcgtggaaggcaaccgtgtcggcgtgagcatagacggtgtaaacctgcctgattctgaagaaaactcgctgtacgcccgttatggcaacttcaacagctcgcgtttgtctatcgaccccgaactcgtgcgcaacatcgaaatcgtgaagggcgcagactctttcaataccggcagtggtgcattgggcggcggtgtgaattaccaaacgctgcaaggccgtgatttgctgttggacgacaggcaattcggcgtgatgatgaaaaacggttacagcacgcgtaaccgtgaatggacaaatactctcggtttcggtgtgagtaacgaccgcgtggatgctgctttgctgtattcgcaacgtcgcggtcatgaaaccgaaagcgcgggaaaccgaggctatgctgtggaaggggaaggcagtggcgcgaatatccgtggttcggcacgcggtatccctgattcgtccaaacacaaataccacagctttttgggtaagattgcttaccaaattaacgataaccaccgcatcggcgcatcgcttaacggccagcagggacataattacacggttgaagagtcttataacctgaccgcttcttcctggcgcgaagccgatgacgtaaacagacggcgcaatgccaacctcttttacgaatggatgcctgattcaaattggttgtcgtctttgaaggcggacttcgattatcagaaaaccaaagtggcggcggttaacaacaaaggctcgttcccgatggattattccacctggacgcgcaactataatcagaaggatttggacgaaatatacaaccgcagcatggacacccgattcaaacgttttactttgcgtttggacagccatccgttgcaactcgggggggggcgacaccgcctgtcgtttaaaactttcgtcagccgccgtgattttgaaaacctaaaccgcgacgattattacttcagcggccgtgttgttcgaaccaccagcagtatccagcatccggtgaaaaccaccaactacggtttctcactgtctgaccaaattcaatggaacgacgtgttcagtagccgcgcaggtatccgttacgaccacaccaaaatgacgcctcaggaattgaatgccgagtgtcatgcttgtgacaaaacaccacctgcagccaacacttataaaggctggagcggttttgtcggcttggcggcgcaactgaatcaggcttggcatgtcggttacgacattacttccggctaccgcgtccccaatgcgtccgaagtgtatttcacctacaaccacggttcgggtaattggctgcctaatcccaacctgaaagccgagcgcagcaccacccacaccctgtctctgcaaggccgcagcgaaaaaggcatgctggatgccaacctgtatcaaagcaattaccgcaatttcctgtctgaagagcagaagctgaccaccagcggcactcccggctgtactgaggaaaatgcttactacggtatatgcagcgacccctacaaagaaaaactggattggcagatgaaaaatatcgacaaggccagaatccgcggtatcgagctgacaggccgtctgaatgtggacaaagtagcgtcttttgttcctgagggttggaaactgttcggctcgctgggttatgcgaaaagcaaactgtcgggcgacaacagcctgctgtccacacagccgctgaaagtgattgccggtatcgactatgaaagtccgagcgaaaaatggggcgtattctcccgcctgacctatctgggcgcgaaaaaggccaaagatgcgcagtacaccgtttatgaaaacaagggctggggtacgcctttgcagaaaaaggtaaaagattacccgtggctgaacaagtcggcttatgtgtttgatatgtacggcttctacaaaccggctaaaaacctgactttgcgtgcaggcgtatataatgtgttcaaccgcaaatacaccacttgggattccctgcgcggcctgtatagctacagcaccaccaactcggtcgaccgcgatggcaaaggcttagaccgctaccgcgccccaagccgtaattacgccgtatcgctggaatggaagttttaa

SEQ ID NO: 19 - protein, 791 aa, Neisseria meningitidis
MKPLQMLPIAALVGSIFGNPVLAADEAATETTPVKAEIKAVRVKGQRNAPAAVERVNLNRIKQEMIRDNKDLVRYSTDVGLSDSGRHQKGFAVRGVEGNRVGVSIDGVNLPDSEENSLYARYGNFNSSRLSIDPELVRNIEIVKGADSFNTGSGALGGGVNYQTLQGRDLLLDDRQFGVMMKNGYSTRNREWTNTLGFGVSNDRVDAALLYSQRRGHETESAGNRGYAVEGEGSGANIRGSARGIPDSSKHKYHSFLGKIAYQINDNHRIGASLNGQQGHNYTVEESYNLTASSWREADDVNRRRNANLFYEWMPDSNWLSSLKADFDYQKTKVAAVNNKGSFPMDYSTWTRNYNQKDLDEIYNRSMDTRFKRFTLRLDSHPLQLGGGRHRLSFKTFVSRRDFENLNRDDYYFSGRVVRTTSSIQHPVKTTNYGFSLSDQIQWNDVFSSRAGIRYDHTKMTPQELNAECHACDKTPPAANTYKGWSGFVGLAAQLNQAWHVGYDITSGYRVPNASEVYFTYNHGSGNWLPNPNLKAERSTTHTLSLQGRSEKGMLDANLYQSNYRNFLSEEQKLTTSGTPGCTEENAYYGICSDPYKEKLDWQMKNIDKARIRGIELTGRLNVDKVASFVPEGWKLFGSLGYAKSKLSGDNSLLSTQPLKVIAGIDYESPSEKWGVFSRLTYLGAKKAKDAQYTVYENKGWGTPLQKKVKDYPWLNKSAYVFDMYGFYKPAKNLTLRAGVYNVFNRKYTTWDSLRGLYSYSTTNSVDRDGKGLDRYRAPSRNYAVSLEWKF

SEQ ID NO: 20 - protein, 147 aa, Neisseria meningitidis
ADEAATETTPVKAEIKAVRVKGQRNAPAAVERVNLNRIKQEMIRDNKDLVRYSTDVGLSDSGRHQKGFAVRGVEGNRVGVSIDGVNLPDSEENSLYARYGNFNSSRLSIDPELVRNIEIVKGADSFNTGSGALGGGVNYQTLQGRDL

SEQ ID NO: 21 - protein, 621 aa, Neisseria meningitidis
LLDDRQFGVMMKNGYSTRNREWTNTLGFGVSNDRVDAALLYSQRRGHETESAGNRGYAVEGEGSGANIRGSARGIPDSSKHKYHSFLGKIAYQINDNHRIGASLNGQQGHNYTVEESYNLTASSWREADDVNRRRNANLFYEWMPDSNWLSSLKADFDYQKTKVAAVNNKGSFPMDYSTWTRNYNQKDLDEIYNRSMDTRFKRFTLRLDSHPLQLGGGRHRLSFKTFVSRRDFENLNRDDYYFSGRVVRTTSSIQHPVKTTNYGFSLSDQIQWNDVFSSRAGIRYDHTKMTPQELNAECHACDKTPPAANTYKGWSGFVGLAAQLNQAWHVGYDITSGYRVPNASEVYFTYNHGSGNWLPNPNLKAERSTTHTLSLQGRSEKGMLDANLYQSNYRNFLSEEQKLTTSGTPGCTEENAYYGICSDPYKEKLDWQMKNIDKARIRGIELTGRLNVDKVASFVPEGWKLFGSLGYAKSKLSGDNSLLSTQPLKVIAGIDYESPSEKWGVFSRLTYLGAKKAKDAQYTVYENKGWGTPLQKKVKDYPWLNKSAYVFDMYGFYKPAKNLTLRAGVYNVFNRKYTTWDSLRGLYSYSTTNSVDRDGKGLDRYRAPSRNYAVSLEWKF

SEQ ID NO: 22 - protein, 768 aa, Neisseria meningitidis
ADEAATETTPVKAEIKAVRVKGQRNAPAAVERVNLNRIKQEMIRDNKDLVRYSTDVGLSDSGRHQKGFAVRGVEGNRVGVSIDGVNLPDSEENSLYARYGNFNSSRLSIDPELVRNIEIVKGADSFNTGSGALGGGVNYQTLQGRDLLLDDRQFGVMMKNGYSTRNREWTNTLGFGVSNDRVDAALLYSQRRGHETESAGNRGYAVEGEGSGANIRGSARGIPDSSKHKYHSFLGKIAYQINDNHRIGASLNGQQGHNYTVEESYNLTASSWREADDVNRRRNANLFYEWMPDSNWLSSLKADFDYQKTKVAAVNNKGSFPMDYSTWTRNYNQKDLDEIYNRSMDTRFKRFTLRLDSHPLQLGGGRHRLSFKTFVSRRDFENLNRDDYYFSGRVVRTTSSIQHPVKTTNYGFSLSDQIQWNDVFSSRAGIRYDHTKMTPQELNAECHACDKTPPAANTYKGWSGFVGLAAQLNQAWHVGYDITSGYRVPNASEVYFTYNHGSGNWLPNPNLKAERSTTHTLSLQGRSEKGMLDANLYQSNYRNFLSEEQKLTTSGTPGCTEENAYYGICSDPYKEKLDWQMKNIDKARIRGIELTGRLNVDKVASFVPEGWKLFGSLGYAKSKLSGDNSLLSTQPLKVIAGIDYESPSEKWGVFSRLTYLGAKKAKDAQYTVYENKGWGTPLQKKVKDYPWLNKSAYVFDMYGFYKPAKNLTLRAGVYNVFNRKYTTWDSLRGLYSYSTTNSVDRDGKGLDRYRAPSRNYAVSLEWKF

SEQ ID NO: 23 - protein, 915 aa, Neisseria meningitidis
MQQQHLFRFNILCLSLMTALPAYAENVQAGQAQEKQLDTIQVKAKKQKTRRDNEVTGLGKLVKSSDTLSKEQVLNIRDLTRYDPGIAVVEQGRGASSGYSIRGMDKNRVSLTVDGVSQIQSYTAQAALGGTRTAGSSGAINEIEYENVKAVEISKGSNSVEQGSGALAGSVAFQTKTADDVIGEGRQWGIQSKTAYSGKNRGLTQSIALAGRIGGAEALLIHTGRRAGEIRAHEDAGRGVQSFNRLVPVEDSSNYAYFIVKEECKNGSYETCKANPKKDVVGKDERQTVSTRDYTGPNRFLADPLSYESRSWLFRPGFRFENKRHYIGGILEHTQQTFDTRDMTVPAFLTKAVFDANKKQAGSLPGNGKYAGNHKYGGLFTNGENGALVGAEYGTGVFYDETHTKSRYGLEYVYTNADKDTWADYARLSYDRQGIGLDNHFQQTHCSADGSDKYCRPSADKPFSYYKSDRVIYGESHRLLQAAFKKSFDTAKIRHNLSVNLGFDRFGSNLRHQDYYYQHANRAYSSNTPPQNNGKKISPNGSETSPYWVTIGRGNVVTGQICRLGNNTYTDCTPRSINGKSYYAAVRDNVRLGRWADVGAGLRYDYRSTHSDDGSVSTGTHRTLSWNAGIVLKPTDWLDLTYRTSTGFRLPSFAEMYGWRAGVQSKAVKIDPEKSFNKEAGIVFKGDFGNLEASWFNNAYRDLIVRGYEAQIKDGKEEAKGDPAYLNAQSARITGINILGKIDWNGVWDKLPEGWYSTFAYNRVRVRDIKKRADRTDIQSHLFDAIQPSRYVVGLGYDQPEGKWGVNGMLTYSKAKEITELLGSRALLNGNSRNTKATARRTRPWYIVDVSGYYTVKKHFTLRAGVYNLLNYRYVTWENVRQTAGGAVNQHKNVGVYNRYAAPGRNYTFSLEMKF

SEQ ID NO: 24 - protein, 712 aa, Neisseria meningitidis
MNNPLVNQAAMVLPVFLLSACLGGGGSFDLDSVDTEAPRPAPKYQDVFSEKPQAQKDQGGYGFAMRLKRRNWYPQAKEDEVKLDESDWEATGLPDEPKELPKRQKSVIEKVETDSDNNIYSSPYLKPSNHQNGNTGNGINQPKNQAKDYENFKYVYSGWFYKHAKREFNLKVEPKSAKNGDDGYIFYHGKEPSRQLPASGKITYKGVWHFATDTKKGQKFREIIQPSKSQGDRYSGFSGDDGEEYSNKNKSTLTDGQEGYGFTSNLEVDFHNKKLTGKLIRNNANTDNNQATTTQYYSLEAQVTGNRFNGKATATDKPQQNSETKEHPFVSDSSSLSGGFFGPQGEELGFRFLSDDQKVAVVGSAKTKDKPANGNTAAASGGTDAAASNGAAGTSSENGKLTTVLDAVELKLGDKEVQKLDNFSNAAQLVVDGIMIPLLPEASESGNNQANQGTNGGTAFTRKFDHTPESDKKDAQAGTQTNGAQTASNTAGDTNGKTKTYEVEVCCSNLNYLKYGMLTRKNSKSAMQAGESSSQADAKTEQVEQSMFLQGERTDEKEIPSEQNIVYRGSWYGYIANDKSTSWSGNASNATSGNRAEFTVNFADKKITGTLTADNRQEATFTIDGNIKDNGFEGTAKTAESGFDLDQSNTTRTPKAYITDAKVQGGFYGPKAEELGGWFAYPGDKQTKNATNASGNSSATVVFGAKRQQPVR

SEQ ID NO: 25 - protein, 186 aa, Neisseria meningitidis
MNMKTLLALAVSAVCSVGVAQAHEHNTIPKGASIEVKVQQLDPVNGNKDVGTVTITESNYGLVFTPDLQGLSEGLHGFHIHENPSCEPKEKEGKLTAGLGAGGHWDPKGAKQHGYPWQDDAHLGDLPALTVLHDGTATNPVLAPRLKHLDDVRGHSIMIHTGGDNHSDHPAPLGGGGPRMACGVIK